(19) United States
(12) Patent Application Publication
(10) Pub. No.: US 2010/0069889 A1
(43) Pub. Date: Mar. 18, 2010

(54) Intelligent medical device system for integrated diagnostics and therapeutics

(76) Inventor: Neal Solomon, Oakland, CA (US)

Correspondence Address: Neal Solomon, P.O. Box 21297, Oakland, CA, 94620, US

(21) Appl. No.: 12/462,784

(22) Filed: Aug. 7, 2009

Related U.S. Application Data
(60) Provisional application No. 61/188,374, filed on Aug. 8, 2008.

(51) Int. Cl. A61M 5/00

(52) U.S. Cl. 604/890.1

(57) ABSTRACT

The system integrates medical diagnostics with therapeutics in an intelligent medical device (iMD) system. Solution options are generated and evaluated by the iMD's diagnostic module, which forwards a custom solution to the therapeutics module. The therapeutics module administers a customized combination of drugs to solve a specific set of pathologies on demand. The system automatically monitors the therapeutic operation, assesses and analyzes the biological system feedback, refines the drug combination, and delivers the refined drugs to the patient.

### CROSS-REFERENCES TO RELATED APPLICATIONS

The present application claims the benefit of priority under 35 U.S.C. §119 from U.S. Provisional Patent Application Ser. No. 61/188,374, filed on Aug. 8, 2008, the disclosure of which is hereby incorporated by reference in their entirety for all purposes.

### FIELD OF THE INVENTION

The invention involves micro electro mechanical systems (MEMS) applied to medical devices and components. The present system includes devices and components for lab on a chip (LOC) apparatus. The invention applies to diagnostic and therapeutic aspects of medical intervention.

### BACKGROUND

As scientists discover the mechanics of genetic processes, our understanding of the sources of diseases increases. The benefits of understanding genetic dynamics and proteomics regulatory processes assists in development of a new generation of medical devices able to diagnose, regulate, manage and cure complex diseases. The potential exists to develop personalized drug therapies to target specific genetic pathologies.

Regarding diagnostic systems, MEMS is an umbrella for a class of new medical devices able to identify genetic mutations and proteomic dysfunctions. While largely external in vitro devices, DNA microarrays, RNA microarrays and protein microarrays provide feedback to identify an individual's genetic information. Protein microarrays use antibodies to assess protein functional responses. In addition, whole cell assays test cells with analytes to assess specific responses to chemical inputs. Multi-phenotype cellular arrays are used for bio-sensing of specific inputs in order to study cell functions.

Though DNA, RNA, protein and whole cell arrays have developed separately, a new generation of lab on chip (LOC) and micro-total analysis systems (μTAS) technologies have emerged as well that integrate several functions in a single device. These multi-purpose arrays provide clinical diagnostic data to practitioners.

In addition to these external devices, the evolution of radiological diagnostic tools has provided a revolution to analytical practitioners. In particular, the use of CT, PET and MRI technologies provides detailed data on specific disease progression. In addition to these external radiological diagnostic technologies, the internal sensing “pill” camera records and transmits digital images to substitute for the surgical intervention of exploratory surgery. Finally, the use of implanted sensors assists in the regulation of simple deterministic expert systems.

The convergence of nanotechnology with biology has produced “bionano” devices. In the main, the use of nanotechnology is limited to particles that are targeted to specific tissue in order to identify pathology and, when combined with directed radiation, provide a therapeutic alternative. The advent of self-assembled peptide nano-biomaterials provides interesting opportunities for diagnostics and therapeutics. The use of nano-scale devices, in which collective behaviors are controlled for therapeutic as well as diagnostic modes, provides an advancement of the bionano field.

Regarding therapeutic medical devices and systems, the field has evolved from the development of the hearing aid and the cardiac pace maker. For instance, the implantable brain pacemaker has been developed to regulate epileptic energy pulses and blood glucose monitoring is regulated with an insulin pump. Moreover, implantable pain management devices are used to control chronic pain. Microfluidic devices to target drug delivery, primarily using a deterministic expert system control model, have also been developed. All of these devices are simple single-function mechanisms targeted to a specific disease or disorder.

An emerging scientific field is providing a new set of technologies from bio-inspired computing. Complexity science deals with self-organizing systems that learn in indeterministic environments. The inspiration from the autonomic nervous system and the human immune system provide computing systems that emulate these complex biological processes. Autonomic computing self-diagnoses, self-heals and self-regulates distributed networks. The human immune system provides inspiration for immunocomputing models that emulate protein regulatory network behaviors in order to solve complex optimization problems. Swarm intelligence metaheuristics provides solutions to optimization problems as well. For instance, the ant colony optimization (ACO) metaheuristic provides a model to solve network computing problems. These models share the ability to develop solutions to problems in self-organizing systems, including plasticity behaviors, in indeterministic environments. In effect, these complex computing and control systems learn. So far, these complex computing models have not been applied to medical devices.

The ability to use genetic and proteomic information to solve complex pathologies provides a new generation of opportunities to build medical devices that are customized to each individual's specific disease(s). Our understanding of cancer, for instance, as the combination of multiple genetic mutations, suggests that each disease type is classed into a typology that can be solved with specific targeted therapies. Given this new knowledge, it is logical to build medical devices that are personalized to specific diseases of each individual. In particular, the use of medical devices focused on solving problems involving pathologies associated with cardiovascular, neurological, immunological and endocrinological systems, and with cancer, is a next step.

Each of the prior medical devices has limitations. For the most part, none of the implantable medical devices are “intelligent”. Rather, they are simple deterministic systems. They are also single function devices focused on a specific narrow medical problem. Because they are merely deterministic expert systems, they do not combine diagnostic and therapeutic functionality. In the diagnostic mode, they do not provide sophisticated modeling functions. Further, prior MDs are not networked since they typically involve a single device performing a single function. Finally, these devices are not useful in personalized medicine, which require complex analysis and targeting of individual therapies to unique problem sets.

What is needed? We need active intelligent medical devices that are able to work with other medical devices to solve multiple medical problems. We need complex medical devices that are capable of integrating diagnostics and therapeutics in order to maximize efficiency, to promote early detection and treatment and to modify functionality with feedback mechanisms to solve complex biological optimization problems in biological regulatory networks. The present system develops an intelligent multifunctional medical device system.

### PROBLEMS THAT THE SYSTEM SOLVES

The present system solves a range of problems. How can we develop an intelligent medical device (iMD) that coordinates diagnosis and therapy? How can the iMD coordinate sensors and integrated circuits? How is the processing of chemical and biological fluids administered by using the iMD? How is the implantable iMD coordinated with external computation and modeling? How does the device collect samples and data in real time? How does one integrate multi-functionality into an efficient iMD design? How is the implantable device installed with minimal invasiveness? How are nano-components integrated into the iMD? How does the iMD use sensors and probes for maximum effect? How does the iMD efficiently analyze biological data? How are solutions to complex problems developed and refined in the iMD? How is drug delivery optimized in the iMD? How can we construct customized drugs for therapies to individual patient pathologies? How can an iMD self-organize and adapt to indeterministic environmental conditions? How can multiple iMDs be coordinated, particularly for multiple applications? Solving these problems presents opportunities to develop a new generation of highly effective medical devices.

### SUMMARY OF THE INVENTION

The iMD system integrates diagnostic and therapeutic operations. After novel solution options are modeled to solve complex multi-objective optimization problems by the modeling processes of the diagnostics module, the therapeutics module organizes a specific combination of drugs and biologicals to apply the solution. The selected therapeutic solution is then assessed and analyzed by the iMD with assistance from on-site probes and sensors. This feedback is evaluated by the diagnostics module and the solution options are modified and refined. When more than one iMD is used, the iMD network simultaneously tracks and manages multiple diseases in various stages of development.

The system automates the personalized medicine model to rapidly solve medical pathologies. The iMD network self-organizes the multiple diagnostic and therapeutic components to track, anticipate and treat the progression of diseases.

### Novelties

The iMD system integrates customized pathology problem solving and therapeutic operations to automate complex self-organized medical treatment processes. This aspect of the iMD system allows the iMD network to customize and automate personalized medicine to solve complex problems in real time.

### ADVANTAGES OF THE INVENTION

The invention allows the integration of diagnostics with therapeutics, thereby increasing the efficiency of the therapeutic modality. The integrated device allows the tracking of therapies by assessing feedback processes in order to more effectively manage complex regulatory networks.

### DESCRIPTION OF THE INVENTION

### (I) Diagnostics and Therapeutics

(1) Assessing diagnostic and therapeutic solution options with iMD system

Most diseases, particularly genetic diseases, have a progression of symptoms. This progression of phases of the disease cycle reflects the continuing degradation of the genetic conditions. The discovery of a disease often is confirmed by the detection of a precursor, such as a malformed protein or an antibody that reacts to a mutated protein in a protein regulatory network. The discovery of each disease type must thus be seen in term of disease cycle assessment.

Through the diagnostic process, disease symptoms lead to discovery of systemic and genetic causes. While radiological diagnostic tools—MRI, CT and PET imaging technologies—are a useful initial step in identifying abnormalities, they require more sophisticated analytical tools for discovery of pathologies at the molecular level. DNA, RNA, protein and whole cell analyses are useful to identify the specific combination of dysfunctional molecular components of pathologies. Lab on a chip (LOC) microarray analyses also provide rapid discovery of multiple molecular mutations that are the source of diseases. From these analyses it is possible to clearly identify the problems that cause numerous pathologies.

Once the cells, DNA and proteins are collected and analyzed, it is necessary to model the unique combinations of mutated genes and dysfunctional proteins that cause each individual's unique type of pathology. These modeling procedures are conducted by internal iMD computing resources in the diagnostic module(s) that are focused on a specific pathology and protein regulatory network pathway as well as by external computation resources. The challenge of modeling is to discover the precise parameters of an individual's multi-objective optimization problems (MOOPs) that manifest as a particular disease. Finding and implementing the solution to MOOPs presents the main challenge of the pathology discovery and therapeutic processes.

In some cases, in order to identify the MOOPs that constitute disease parameters it is necessary to interact with the biological system to create states that yield evidence that will clearly model the system. This interaction process during the disease discovery course may involve inducing an intervention in order to track a regulatory network response. From feedback with the biological system, the iMDs are able to more rapidly and accurately diagnose a MOOP that constitutes a disease state.

In this approach, the modeling system begins with a set of hypotheses about the nature of the disease and develops a series of tests that include the active intervention with the complex biological system it is diagnosing. Because it is on-site, the iMD system is uniquely suited to develop the interaction process that allows this immediate diagnostic course to maximize efficiency.

The interactive approach to disease diagnosis is integrated with solution options as well. By providing therapeutic options from among a range of options to solve the MOOPs, the system is able to refine the solution and cure the disease. The present system advances this approach to disease remedies by integrating diagnostic and therapeutic components to solve pathologies.

### (II) Drug Delivery

(2) Method for Drug Delivery Application with IMDs

IMDs have multiple compartments to store chemicals. There are three types of drug and agent categories that the iMD administers and regulates. First, the iMD administers standard drugs that are available in any traditional pharmacy. Second, the iMD administers personalized medicines, such as the synthesized proteins, that are constructed for each patient's unique pathologies. Finally, the iMD administers synthetic drugs and biologicals that it combines on-site to solve a specific pathology.

Regarding the first class of drugs, the iMD uses drugs that target specific system pathologies. For neurological pathologies, the system uses drugs that are traditionally used for psychopathologies and neuroendocrinological, CNS and neurovascular disorders. For cancer, the system uses chemotherapies. For cardiovascular diseases, the system uses drugs that treat blood pressure, inflammation and cholesterol.

Regarding the second class of drugs, the iMD uses custom designed drugs that treat specific dysfunctional proteins that are created by mutated genes. In some cases, the use of the precise characterization of a drug requires existence of a specific gene in order to be effective. In other cases, the iMD uses interactive procedures so as to actively test for the existence of specific genetic mutations and models the dysfunction in order to produce a customized drug.

Regarding the third class of drugs, the iMD uses an active process to synthesize a “drug” on-site by using the patient's own affected cells, filtering the cells on-board, treating the cells and outputting the cells to affect a treatment remedy. The cells may be treated either with gene replacement therapy, RNAi therapy or the filtration of mutated proteins. This cellular replacement therapy allows the iMD to behave as an internal factory in an active therapeutic way. In combination to its diagnostic functions, this active therapeutic function allows the iMD to advance personalized interactive medicine.

### (3) Therapeutic Modalities of IMDs

There are three therapeutic goals advanced by the iMD system. First, the aim of the iMD is to destroy a disease. Second, the goal is to manage or control a disease and to limit or stop its progression. Third, the goal is to prevent a disease from manifesting. There are different ways of achieving each of these goals in different disease situations.

In order to achieve these therapeutic goals, iMDs are useful for advancing several main therapeutic modalities:
(a) Administer a drug
(b) Administer a combination of synthetic proteins to limit dysfunctional protein behaviors
(c) Administer cell receptor inhibition (to block a dysfunctional protein function)
(d) Administer RNAi to block dysfunctional protein generation
(e) Administer stem cells
(f) Block a gene
(g) Block the blood supply feeding a tissue (angiogenesis) [for neoplasties]
(h) Target radiation to a specific tissue location [for neoplasties]
(i) Prevent formation of, or to track down and destroy, cellular metastases [for neoplasties]
(j) Administer gene therapy by inserting a gene into DNA of cells to obtain a new protein expression (using various methods)
(k) Administer protein therapy
(l) Remove patient's cells, treat with one of the methods above, and reinsert the cells in select tissue
(m) Some combination of these modalities

So far, most traditional medical device therapies have focused on (a) above. However, the multiple therapeutic modalities presented here allow the iMD to initiate dramatic medical breakthroughs. By implementing therapeutic strategies for activating and deactivating specific genes on demand, the system solves complex pathologies. These strategies, however, work precisely by having the on-site interactive capabilities that require implementing feedback mechanisms.

By employing several iMDs in a network, the present system actively solves biological MOOPs in multiple pathologies simultaneously.

### (4) Therapeutic Goal Evaluation Procedure Using IMDs

The present system allows iMDs to present solution options to diagnostic hypotheses of MOOPs that manifest as pathology. The interactive feature of integrating iMD diagnostic and therapeutic functions allows the present system to solve complex biological problems on the fly with constantly refined solutions. As more evidence is accumulated from monitoring the protein regulatory networks and genetic conditions, the modeling components of the diagnostic element is able to actively track specific therapeutic modality effectiveness. If one approach is effective, the system will continue to develop this track of therapy. If an approach is ineffective, this evidence is input into the model in the continued search for a cure. The active therapeutic solutions are input into the iMD databases for later retrieval of solutions to similar problems.

As the feedback from therapeutic solution attempts are interpreted, the iMD system modulates the therapeutic strategy between the main goals of pathology destruction, pathology management and pathology prevention. As it is clarified that a set of therapies has limited effectiveness on pathology destruction, the iMD shifts to a pathology management priority. For example, in the case of controlling some types of cancer, specific combinations of genetic mutations may require a pathology management regimen, such as protein replacement therapy that is limited to a single cell cycle, rather than the expectation of pathology destruction. Further, by modeling the disease cycle, the iMD may seek to prevent a pathology progression, the condition for which it anticipates in multiple scenarios, so as to minimize the probable expected disease manifestation.

### (5) Procedure for Targeting Cells for Drug Delivery Using Antibodies, Proteins and Nanorobotic Collectives in IMD System

There are several main ways to target drugs to specific cells. First, monoclonal antibodies are used to target specific cells. Second, the system uses specific proteins that target tissues. Third, the system uses drug-carrying nanorobotic collectives that target specific cells. Finally, engineered viruses deliver proteins or antibodies to specific cells. These four methods of targeting cells for drug delivery are used by the iMD system.

One of the most effective methods of precisely targeting drug therapies involves the use of monoclonal antibodies. Antibodies are combined with a specific drug therapy to deliver the drug to a specific cell type that is affected by a genetic mutation.

The discovery that specific proteins in the vasculature have different “zip codes” identifying their locations allows the present system to use specific proteins that refer to different locations in the body to combine with active therapeutic proteins. In effect, these proteins are “tuned” to a specific frequency of the targeted cells.

The use of nanorobots to precisely target cells is used to deliver drugs as well. Intelligent nanorobots use computational and navigational components to guide the collective to a specific target.

Engineered viruses are also used to target genes, proteins and nanorobots to specific cells. For example, a virus transfers a T cell receptor gene to a T cell, which then trains the T cell to seek out and destroy a tumor.

These targeting mechanisms are used by activating specific compartments in the iMD that contain antibodies, nanorobots, targeting proteins and engineered viruses. Once the mutated cells are identified by using the diagnostic process, the iMD accesses the compartment containing one of these targeting mechanisms and combines the antibody, nanorobot, engineered virus or targeting protein with a drug therapy solution. The targeting mechanisms then bring the drug to the precise cellular locations to treat the tissue.

In some cases, the combination of these three targeting models is used by the iMD system, for instance, by placing the drugs as cargo in the nanorobots for targeting by the antibodies, or by carrying synthetic proteins in engineered viruses.

An example of the use of these precise targeting mechanisms by the iMD system lies in the case of metastatic cancer in which cells from tumor cells spread from the initial cancer site. Tracking these cells is complex. In fact, since the human immune system typically tracks tumor cells it is an elegant solution to tune or train antibodies to track down metastatic cancers. In this same way, the iMD system “tunes” the antibodies to specific frequencies by seeding them with the affected cells or with specific proteins.

Because the system solves multiple pathologies simultaneously, the iMD may use the several types of targeting mechanisms, or combinations, at the same time.

### (III) Combining multiple drugs

(6) System for on-Site Pharmacy Using IMDs

The iMD contains several compartments for storage of chemicals and biological entities on a layer of the therapeutic module. On one layer of the iMD are compartments for cell samples, for DNA and RNA samples, for specific classes of proteins, for antibody samples, for nanorobotic collectives, for viruses and for stem cells. On another layer of the iMD are compartments for traditional drugs and for customized drugs. The iMD apparatus uses the conduits and plumbing system to combine specific molecules, cells and chemicals in order to activate therapeutic strategies. Depending on the specific pathologies that are being solved by the iMD, the location of the compartments containing the molecules, cells and chemicals are variable.

The storage of chemicals or biologicals is maintained in separate chambers on the periphery of each layer. As a specific set of chemicals are required to be combined, the chemicals are released at a regular rate and mixed in the middle chambers of the layer. The newly mixed chemicals are then sent to another layer for combination with biologicals. For instance, a custom combination of chemicals is loaded on to a nanorobotic collective, which is then dipped in proteins and antibodies. In other examples, stem cells are combined with viruses and specific gene sequences for application to a patient's cells. Each layer has a configuration of partitions through which each different set of chemicals travels. As the doors of one chamber open, they enable the mixture of one chemical in an adjacent chamber. The structure of partitions on each layer is also reconfigurable. This model allows the iMD to perform customized tasks on demand.

This revolutionary model of drug activation for pathology therapy has the advantage of immediate responsiveness to a disease progression. By using antibodies and nanorobots, the delivery vehicles are available for direct targeting to cells. The compartments for cell, DNA and RNA samples provide an on-board iMD storage facility for microarray analysis for immediate diagnostic capabilities. The storage and use of stem cells provides for cell replacement therapies. Finally, storage of customized proteins allows the unique combination of proteins to solve the problem of dysfunctional proteins generated by specific combinations of mutated genes.

The iMD system combines the cells, molecules and/or drugs in specific combinations contingent on the outcome of the diagnostic solution recommendations for a specific disease therapy regimen.

Further, the use of multiple iMDs in a network provides extra capacity for multiple cells, modules and drugs. The ability to share resources in the iMD network allows a single iMD to store only specific types of drugs, while others store other biological entities. Moreover, because this system is linked with a network of tubes, the system can restructure its storage capabilities to switch locations of specific biological and chemical entities based on the needs of the system. As a disease changes its progression, addressing another disease requires a transformation of storage system and the locations of the drugs and biological entities. This model extends the application parameters of the iMD system appreciably.

### (7) Process for Optimizing Combinatorial Chemistry Organization and Distribution in IMD System

The iMD system allows multiple chemicals to be combined on demand. Different chemicals and proteins are located in specific compartments on the iMD. The iMD uses its microfluidic conduit network to combine specific chemicals in specific chambers for later use in targeting specific cells. Once the diagnostic module analyzes a protein dysfunction and a genetic mutation, the diagnostic model proposes a specific set of therapeutic solution options.

The solution options are sent to the therapeutic module for initiating the process of combining specific chemicals and biological entities in order to solve the pathology. The chemicals and biologicals are then mixed in one of the empty iMD chambers to produce a customized concoction that is then sent to a new compartment for combination with the delivery vehicle (an antibody, protein, virus or nanodevice).

(8) System for Mini-Factory to Produce Medicinal Outputs from Multiple Diagnostics in IMD

In general, once the diagnostic module identifies the unique combination of genetic mutations and the set of corresponding dysfunctional proteins, the therapeutic module creates a unique customized medicinal solution to solve the combinatorial optimization problem. The customized solution is then delivered to specific cells.

After the customized solution is delivered, the system obtains feedback from the cells and diagnoses the progress of the solution. This process of continued diagnoses allow the system to refine the solution options and to create new and different customized solutions that take advantage of the feedback, as well as new information used in the interim, in the diagnostic model. This procedure continues until the therapy is refined and the pathology progression achieves a state of equilibrium.

This operational model suggests that the iMD is a mini-factory that generates original medicinal outputs from novel diagnostics and continues to refine therapeutic solution vectors.

When combined with multiple iMDs in a network the system operates like a supply chain, with multiple chemicals dispersed in different locations. When a demand is made for a chemical, it is shifted from one location to another until the chemical is combined into a customized therapy. Since most iMDs are located in specific places that focus on specific pathologies of specific biological systems, each iMD is routinely used autonomously. However, the combination of solutions to multiple simultaneous pathologies provides an extremely novel system in the development of medicinal therapeutics.

### (IV) Personalized Medicine

### (9) System for Semi-Personalized Medicine Using IMDs

Many genetic diseases are caused by identification of multiple specific mutated genes. For example, lymphoma and breast cancers are classified into a typology of dozens of individual categories of tumors that are generated from different combinations of mutated genes. By classifying these specific combinations of gene mutation types, it is possible to project the aggressiveness of the successive pathology.

Semi-personalized medicine uses these gene mutation combination classification schemata to develop specific solutions targeted to the genes or to the dysfunctional proteins that are generated by the mutated genes. Rather than target a specific individual's precise combination of gene mutations, which would be timely and costly, semi-personalized drug therapies target the most common mutation combinations.

IMDs store specific drugs that are targeted to a specific combination of mutations that are identified by prior genetic testing. The iMD therapeutic module administers the drugs that target a specific class of gene mutation combinations.

### (10) System for Active Personalized Medicine Using IMDs

The iMD system is used to develop an active personalized medical regimen. After the initial diagnosis of a pathology based on an analysis of a specific combination of genetic mutations, the therapeutic module combines a set of chemicals and proteins to solve the MOOP. The drug combination is then targeted to the affected cells.

The system obtains sensor feedback from the resultant medicinal solution application. In some cases, the iMD collects a cell sample to analyze the initial solution. The sensor data and the cell samples are then analyzed and the diagnostic model is updated. The treatment regimen is modified by the therapeutic module by accessing the updated model in order to improve a solution. Again, the new solution is assessed and modified and the system applies refined solutions until the problem is solved.

The use of multiple iMDs is particularly efficient in their application to personalized medical solutions. While one or more iMDs focus on the diagnostic element, the others focus on the therapeutic element. Multiple diagnostic and therapeutic functions occur simultaneously with multiple iMDs. This process uses a division of labor to maximize the efficiency of the overall system to accelerate results.

Using multiple iMDs also provides ample reserves of multiple chemicals that extend the parameters of possible drug combinations.

(11) Method for Integrating Imaging Diagnostics with Active IMDs

In another model of implementing active personalized medical protocols using iMDs, imaging diagnostic tools identify and assess an initial disease condition. Once the initial condition is identified, the iMD is activated and a unique combination of chemicals is generated to target the pathological tissue. The drug is then delivered and the iMD tracks the therapy with the diagnostic module. The iMD collects diagnostic data and forwards the data to an external computer system for extensive modeling. The iMD system then develops an updated drug combination regimen based on the new data from the modeling analyses of the disease progression and a further genetic mutation analysis of the outcome of the therapy. The updated medicinal solution option is then delivered to the affected tissue. The process continues until the problem is managed or solved.

### (12) Method for Screening Dysfunctional Proteins in Filtering Process Using IMDs

One important therapeutic modality employed by iMDs is protein replacement therapy (PRT). The PRT procedure used by the iMDs begins by collecting a patient's affected proteins and filters the proteins to remove the dysfunctional proteins. The healthy proteins are returned to the affected cells. The dysfunctional proteins are “fortified” with peptides that are healthy. The synthesized proteins are then returned to the affected cells.

In another model of the PRT process, the iMD collects affected cells and screens the dysfunctional proteins from the cells. The iMD then replaces the affected proteins with healthy proteins from other healthy cell lines.

PRT does not repair DNA or the mutated proteins of affected cells. Rather, this procedure repairs the consequences of the haplotypes which cause the dysfunctional protein configurations. The PRT process must therefore be repeated each cell cycle to repair each successive cell's dysfunctional DNA.

### (V) Drug-Device Combinations with IMD System

### (13) System for Customized Modulation of Therapeutics Using IMDs

The iMD system is a mechanism for drug delivery and feedback that combines diagnostics and drugs with automated hardware and software. These intelligent devices provide the combination of device with biologic, drug with biologic and device with drug in a complex package. The iMD provides customized modulation of therapeutics that allow medicines to be tailored to specific diseases. With the diagnostic module of the iMD, complex optimization problems of disease causes are analyzed and solutions to the problems are provided in the form of customized diagnostics to unique combinations of genetic mutations. Once the system develops an initial map of individual dysfunction, the same iMD develops multiple medicinal therapeutic solutions.

The interactive dimension of the iMD diagnostic and therapeutic system provides a two part functional model that consists of a course-grained initial response and a fine-grained response after the initial feedback. While the iMD is capable of straightforward drug administration, it has the advantage of precise targeting to cells as well as the ability to integrate the diagnostic processes with the therapeutic operations.

### (VI) Intelligent Medical Systems

### (14) System for Integrating Diagnostics and Therapeutics Using IMDs

The integration of diagnostics with therapeutics in iMDs presents a system of experimentation for therapeutic refinement in real time. With the system, feedback is regulated by the information that is obtained after a prior solution is administered. From this model, the iMD system presents a form of learning from experience that guides the continuous adjustment of chemicals and biologicals to meet a goal (i.e., eliminating a disease) or maintaining a pathology equilibrium condition. The continuous optimization process of multiple objectives presents a modular updatable expert system that is customized to solve specific disease situations.

In a further procedure for integrating diagnostics and therapeutics, the delivery mechanism of the drug(s) is itself the probe that performs diagnostics. As the diagnostic device travels from the iMD to the pathology cell site, it performs the therapy and the continuing diagnoses.

In another combination of diagnostics and therapeutics in the present system, tumors are identified by the iMD probes and removed by endoscopic surgical techniques. The remaining surrounding cells are targeted by the iMD with a specific drug to prevent tumor recurrence.

(15) Method for Tracking Interactive Therapeutics in Real Time with IMDs

The tracking process of therapeutic modality applications occurs by the diagnostic element of the iMDs. The iMD presents a continuous recording and storage of the performance of the therapeutic system, similar to an aircraft's black box. As the pathological progression degradation process occurs over time, the iMD tracks the multiple variables of therapeutic procedures as well as the feedback of the biological system. The data from the probes regarding the therapeutic interaction process is input into the iMD's database for rapid access. The on-board database is periodically backed up to the external computer system.

This tracking process is important in order to identify drug interaction problems. The process of accessing drug interactions occurs by inputting the diagnostic recommendations for solutions to MOOPs into the model. The model then checks the database for drug interactions and removes the worst drug element from the recommended regimen.

The combination of multiple iMDs in a network provides additional opportunities to track therapeutic applications. In one approach, a single iMD performs the data collection, modeling and analysis of the diagnostics while other iMDs provide the therapeutic procedures and another iMD specifically tracks the process.

In another approach, all of the iMDs work together to share all of the functions in a distributed network, thereby maximizing the functionality and efficiency of the overall system. The utility of multiple iMDs is to employ a distributed database management system to store and access data among the iMDs in the overall system.

### (16) Therapy Testing Process for Continuous Optimization of Multiple Objectives

While most medical systems provide passive diagnostics and pre-determined therapeutics, the present system provides customized solutions to complex problems. One key element of the iMD system is the learning component of the interaction between the diagnostic and therapeutic applications that presents a modular updatable expert system.

The system is activated by the presentation and identification of a biological pathology, which is succeeded by the diagnostic process of seeking solution options to MOOPs. The therapy experimentation process tests the solution options over time by employing the tracking and evaluation mechanisms of the advanced diagnostic process.

This iMD system provides interactive on-demand medical solution administration for complex pathologies. Consequently, it presents a new generation of tools and techniques for solving medical problems.

Reference to the remaining portions of the specification, including the drawings and claims, will realize other features and advantages of the present invention. Further features and advantages of the present invention, as well as the structure and operation of various embodiments of the present invention, are described in detail below with respect to accompanying drawings.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes in their entirety.

### List of Traditional Medicines Used in IMDs

### Neurological System

Benzodiazepines (Alprazolam, Chlordiazepoxide, Clorazepate, Clonazepam, Diaepam, Estraxolam, Flurazepam, Halazepam, Lorazempam, Midazolam, Oxazepam, Quazepam, Temazepam and Triazolam), barbiturates (Amobarbital, Pentobarbital, Phenobarbital and Secobarbital), and benzodiazepine antagonists (Flumazenil).
Antiseizure drugs (Carbamazepine, Clonazepam, Clorzepate dipotassium, Diazepam, Ethosuximide, Ethotoin, Felbamate, Fosphenyto in, Gabapentin, Lamotrigine, Levetiracetam, Lorazepam, Mephenytoin, Mephobarbital, Oxycarbazepine, Pentobartital sodium, Phenobarbital, Phenytoin, Primidone, Tiagabine, Topiramate, Trimethadione and Valproic acid).
Parkinson's and Huntington's drugs (Amantadine, Denstropine, Biperiden, Bromocriptine, Carbidopa, Entacapone, Levodopa, Orphenadrine, Penicillamine, Pergolide, Pramipexole, Procyclidine, Ropinirole, Selegiline, Tolcapone, Trientine and Trihexyphenidyl).
Antipsychotic agents (Aripirzole, Chlorpromaxine, Clorzapine, Fluphenazine, Fluphenazine esters, Haloperidol, Haloperidol ester, Loxapine, Mesoridazine, Molindone, Olanzapine, Perphenanzine, Pimozide, Prochloriperazine, Promazine, Quetiapine, Risperidone, Thioridazine, Thiothixene, Trifluoperazine, Trifluprommazine, and Ziprasidone).
Mood stabilizers (Carbamazepine, Divalproex, Lithium carbonate and Valprioic acid).

### Antidepressants (Amitriptyline, Amoxapine, Bupriopion, Clomipramine, Desipramine, Doxepin, Imipramine, Maprotiline, Mirtazapine, Nefazodone, Nortriptyline, Protriptyline, Trazodone, Trimipramine and Venlafaxone).

### Serotonin Reuptake Inhibitors (Citalopram, Escitalopram, Fluoxetine, Fluvoxamine, Parosetine and Sertaline).

### Monoamine Oxidase Inhibitors (Phenelzine and Tranylcypromine).

Analgesic Opoids (Alfentanil, Buprenorphine, Butorphanol, Codeine, Dezocine, Fentanyl, Hydromorphone, Levomethadyl acetate, Levorphanol, Meperidine, Methadone, Morphine sulfate, Nalbuphine, Oxycondone, Oxymorphone, Pentazocine, Propoxyphene, Remifentanil, Sufetanil and Tramadol).

### Cardiovascular System

Nitrates and nitrites (Amyl nitrite, Isosorbide dinitrate, Isosorbide mononitrate and nitroglycerin); Calcium channel blockers (diltiazem, Felodipine, Isradipine, Nicardipine, Nifedipine, Nimodipine, Nioldipine and Verapamil).
Adrenoceptor antagonist drugs: Alpha blockers (Doxazosin, Phenosybenzmine, Phentolamine, Prazosin, tamsulosin, Terazosin and Tolazoline); Beta blockers (Acebutolol, Atenolol, Betaxolol, Bisoprolol, Barteolol, Carvedilil, Esmolol, Labetolol, Levoobunolol, Metipranolol, Metoprolol, Nadolol, Penbutolol, Pindolol, Propranolol, Sotalol and Timolol).
Adrenoreceptor activating and sympathomimetic drugs (Aproclonidine, Brimonidine, Dexmedetromidine, Dexmethylphenidate, Dextroamphetamine, Dipivefrin, Dobutamine, Dopamine, Ephedrine, Epinephrine, Fenoldopam, Hydroxyamphetamine, Isoproterenol, Methentermine, Metaraminol, Methamphetamine, Methoxamine, Methylphenidate, Midodrine, Modafinil, Naphazoline, Norepinephrine, Oxymetrazoline, Pemoline, Phendimetrazine, Phenylephrine, Pseudoephedrine, Tetrahyrdozoline and Xylometazoline).
Cholinoceptor-activating and cholinesterase-inhibiting drugs: Direct acting cholinomimetics (Acetylcholine, Bethanechol, Carbachol, Cevimeline and Pilocarpine) and cholinesterage inhibitors (Ambenonium, Demecarium, Donepezil, Echothiophate, Edroponium, Galantamine, Neostigmine, Physostigmine, Pyridostigmine, Rivastigmine and Tacrine).
Antihypertensive agents: Beta adrenoceptor blockers (Acebutolol, Atenolol, Betaxolol, Bisoprolol, Bareolol, Carvedilol, Esmolol, Labetalol, Metoprolol, Nadolol, Penbutolol, Pindolol, Propanolol and Timolol), Cetnrally acting sympthoplegic drugs (Clonidine, Guanabenz, Guanfacine and Methyldopa), Postganglionic sympathetic nerve terminal blockers (Guanadrel, Guanethidine and Reserpine), Alpha selective adrenoceptor blockers (Doxazosin, Prazosin and Terzosin), Vasodilators for Hypertension (Diazoxide, Fenoldopam, Hydralazine, Minoxidil and Ntroprusside) and calcium channel blockers (Amlodipine, Diltiazem, Felodipine, Isradipine, Nicardipine, Nisoldipine, Nifedipine and Verapamil); Angiotensin-converting enzyme inhibitors (Benazepril, Captopril, Enalapril, Fosinopril, Lisonopril, Moexiril, Perindopril, Quinapril, Ramipril and Trandolapril); Angiotensin receptor blockers (Candesaran, Eprosartan, lrbesartan, Losartan, Olmisartan, Telmisartan and Valsartan).
Angiotensin receptor blocker (Candesrtan, Eprosartan, Irbesartan, Losartan, Olmesartan, Telmiisartan and Valsartan).
Cardiac Arrhythmias: Sodium channel blockers (Sidopyramide, Flecainide, Lidocaine, Mexiletine, Moricizine, Procainamide, Propafenone, Quinidine sulfate, Quinidine gluconate and Quinidine polygalacturonate), Calcium channel blockers (Bepridil, Diltiazem and Verpamil).

### Statins (Atorvastatin, Cholestyramine, Colesevelam, Colestipol, Ezetimibe, Fenofibrate, Fluvastatis, Gemfibrozil, Lovastatin, Pravastatin, Rosuvastatin and Simvastatin).

### Cancer Therapies

Cancer chemotherapy drugs (Bleomycin, Dactinomycin, Daunorubicin Docetaxel, Doxorubicin, Etopside, Idarubicin, Irinotecan, Mitomycin, Paclitaxel, Topotecan, Vinblastine, Vincristine and Cinorelbine).
Alkylating chemotherapy agents (Mechlorethamine, Chlorambucil, Cycloprhosphamide, Melphalan, Thiotepa, Busulfan, Carmustine, Lomustine, Altetamine, Procarbazine, Dacarbazine, Cisplatin, Carboplatin and Oxaliplatin).
Antimetabolites for chemotherapy (Capecitabine, Cladribine, Cytarabine, Fludarabine, Fluoruracil, Gemcitabine, Mercaptopurine, Methotrexate and Thioguanine).
Hormonal agents for chemotherapy (Flutamide, Tamoxifen, Megestrol acetate, Hydrocortisone, Prednisone, Goserelin acetate, Leuprolie, Aminoglutethimide, Anastrozole, Exemestane and Letrozole).
Cancer drugs (Erbitus, Gleevec, Herceptin, Rituxan and Tarceva).

### Immune System

Immunopharmacological drugs and agents (Abciximab, Adalimumab, Alefacept, Alemtuzumab, Anti-Thymocyte Globulin, Azathioprine, Basilizimab, Bacillus Calmette-Guerin, Cyclophosphamide, Cyclosporin, Daclizumab, Etanercept, Gemtuzumab, Glatiramer, Ibritumomad tiuxetan, Immune Globulin lntrovenous, Infliximab, Interferon alfa-2a, Interferon alfa-2b, Interferon beta-1A, Interferon beta-1B, Interferon gamma-1b, Interleukin-2, Leflunomide, Levamisole, Lymphocyte immune globulin, Metholyprediisolone sodium succinate, Muromonab-CD3, Mycophenolate mofetil, Pegademase Bovine, Pegiterferon afa-2a, Perinterferon alfa-2b, Prednisone, Rh (D) Immune Globulin Micro-dose, Rituximab, Sirolimus, Tacrolimus, Thalidomid and Trastuzumab).

### Endocrine System

Endocrine drugs (Bromocriptine, Cabergoline, Cetrorelix, Chorionic gonadotropin, Corticoreline ovine, Cortictropin, Cosyntropin, Desmopressin, Follitropin alfa, Follitropin beta, Ganirelix, Gonadorelin acetate, Gonadoreline hyrdrochloride, Goserelin acetate, Histrelin, Leuprolide, Menotropins, Nafarelin, Octreotine, Oxytocin, Pergolide, Protirelin, Sermorelin, Somatrem, Somatropin, Thyrotropin alpha, Triptorelin, Urofollitropin and Vasopressin).
Thyroid Agents (Levothryoxine, Liothyronine and Liotrix) and Anti-Thyroid Agents (Diatrizoate sodium, Iodide, lopanoic acid, Ipodate sodium, Methimazole, Patassium iodide, Propylthiouracil and Thyrotropin).
Glucocorticoids (Betamethasone, Betamethasone sodium phosphate, Cortisone, Dexamethasone, Dexamethasone acetate, Dexamethasone sodium phosphate, Hydrocortisone, Hydrocortisone acetate, Hydrocortisone cypionate Hydrocortisone sodium phosphate Hydrocortisone sodium succinate, Metolyprednisolone, Methlpredmisolone acetate, Methylprednisolone sodium succinate, Prednisolone, Prednisolone acetate, Prednisolone sodium phosphate, Prednisolone tebutate, Prednisone, Triamcinonlone, Triamcinolone acetonide, Triamcinolone diacetate and Triamcinolone hexacetonide).
Estrogens (Conjugated estrogens, Dienestrol, Diethylstilbestrol diphosphate, Esterified estrogens, Estradiol, Estropipate, Ethinyl estradiol), Progestins (Hydroxyprogesterone caproate, Levonorgestrel, Medroxyprogesterone acetate, Megestrol acetate, Norethindrone acetate, Norgestrel and Progestone).
Pancreatic Hormones; Sulfonylureas (Acetohexamide, Chlorpropamide, Glimepiride, Glipizide, Glyburide, Tolazamide and Tolbutamide); Meglitinide drugs (Repaglinde and Nateglinide).
This is not intended to be a complete list of drugs, agents or chemicals for use in the iMDs.

### DESCRIPTION OF THE DRAWINGS

FIG. 1 is a schematic diagram showing a 3D iMD with separate analytical, diagnostic and therapeutic module components.

FIG. 2 is a schematic diagram showing a network of two iMDs interacting with external pathologies simultaneously.

FIG. 3 is a flow chart describing the process of a diagnostic module collecting and analyzing biological and chemical samples.

FIG. 4 is a drawing of an iMD with separate components interacting with patient tissue.

FIG. 5 is a schematic diagram showing a 3D iMD with internal components interacting with specific pathologies.

FIG. 6 is a flow chart describing the process of an iMD initiating therapeutic protocols.

FIG. 7 is a flow chart describing the analytical process of assessing and applying a therapy using an iMD.

FIG. 8 is a flow chart describing the process of identifying cell receptor inhibition and designing a therapy using an iMD.

FIG. 9 is a flow chart describing the process of identifying dysfunctional proteins and applying RNAi solutions using an iMD.

FIG. 10 is a flow chart describing the process of generating models of gene mutations using an iMD.

FIG. 11 is a flow chart describing the process of analysis and solution option generation using an iMD.

FIG. 12 is a flow chart describing the process of developing solution options from diagnostic data and analysis in an iMD.

FIG. 13 is a flow chart describing the process of evolving a solution using the analytical and diagnostic modules of an iMD.

FIG. 14 is a schematic diagram showing two drugs administered by two iMDs to a tissue.

FIG. 15 is a schematic drawing of an iMD simultaneously interacting with two tissues and solving two pathologies with different therapies.

FIG. 16 is a schematic drawing of an iMD interacting with specific tissue locations in the vasculature using vascular zip code proteins.

FIG. 17 is a schematic diagram showing an iMD combining proteins in different therapeutic module chambers for delivery to two different pathologies.

FIG. 18 is a schematic diagram showing a T cell receptor gene transferred to viruses in chambers of two therapeutic modules of an iMD to solve several pathologies.

FIG. 19 is a 3D diagram showing an iMD with multiple compartments in which proteins, viruses and antibodies are combined for treating two simultaneous pathologies.

FIG. 20 is a schematic diagram showing an iMD with multiple compartments in which proteins are combined with antibodies in two therapeutic modules in order to track, attack and destroy metastatic cancer cells.

FIG. 21 is a 3D diagram showing the side view of the therapeutic module with multiple biologicals and chemicals in specific compartments.

FIG. 22 is a schematic diagram showing the compartments of a therapeutic module of an iMD in which proteins, antibodies and a virus are combined and delivered to a cell site for therapy.

FIG. 23 is a flow chart showing the process of combining specific biologicals and chemicals for application to pathologies by using the therapeutic module of an iMD.

FIG. 24 is a schematic diagram showing different delivery vehicles, including a virus, nanodevices and stem cells, applied by a therapeutic module of an iMD simultaneously to different tissues.

FIG. 25 is a schematic diagram showing two therapeutic modules solving several problems simultaneously.

FIG. 26 is a schematic diagram showing the storage refill system mechanism in a network of iMDs.

FIG. 27 is a drawing showing the conduit network of a therapeutic module.

FIG. 28 is a schematic drawing showing the sequence order of combining different elements and mixing all elements for application by a therapeutic module of an iMD for solving a pathology.

FIG. 29 is a schematic diagram showing the order of a therapeutic process with feedback using multiple modules of an iMD.

FIG. 30 is a schematic diagram showing the network operations with satellite refills and multiple parallel operations using two iMDs to solve two pathologies simultaneously.

FIG. 31 is a flow chart showing the process of semi-personalized medicine using an iMD.

FIG. 32 is a flow chart showing the process of personalized medicine using an iMD.

FIG. 33 is a flow chart showing the process of active personalized medicine using an iMD.

FIG. 34 is a schematic diagram showing the process of personalized medicine using two iMDs to solve several pathologies simultaneously.

FIG. 35 is a schematic diagram showing an iMD interacting with a pathology by using external computation for analysis after using an imaging diagnostic tool.

FIG. 36 is a flow chart showing the process of using the iMD diagnostic and therapeutic modules to acquire and filter a patient's proteins for protein replacement therapy.

FIG. 37 is a schematic diagram showing the sequence of therapy from course-grained to fine-grained response to diagnosis and analysis by using feedback of therapy to update the analytical model to refine solutions to pathologies using the iMD.

FIG. 38 is a flow chart describing the process of therapeutic assessment and refinement by updating model and solution options using an iMD.

FIG. 39 is a schematic diagram showing how probes perform diagnostics and apply therapy at two cell sites simultaneously using an iMD.

FIG. 40 is a schematic diagram showing probes from an iMD identifying a tumor, which is then extracted endoscopically, and targeting of the surrounding tissue with the therapeutic module of the iMD.

FIG. 41 is a flow chart describing the process of a diagnostic module and a analytical module tracking a remedy, assessing and comparing drug interactions to a database and modifying the remedies.

FIG. 42 is a flow chart describing the process of several iMDs working together to apply and track remedies to several pathologies.

FIG. 43 is a schematic diagram showing an iMD tracking the performance of several other iMDs as they solve several simultaneous pathologies.

FIG. 44 is a flow chart describing the therapy testing and optimization process.

FIG. 45 is a table showing the iMD module categories and functions.

### DETAILED DESCRIPTION OF THE DRAWINGS

The diagnostic, analytical and therapeutic modules of an iMD work together to identify pathology, analyze the pathology and develop remedies to manage or eliminate the pathology. While the diagnostic module collects biological samples and provides an analysis of the samples, and the analytical module supplies complex modeling of the pathology and develops solution options and the therapeutic module combines and applies remedies to solve medical problems. The therapeutic module relies on the data from the diagnostic and analytical modules to identify problems, but the therapeutic module is the critical link in organizing and reorganizing solution options to effectively manage diseases. Each iMD typically contains at least two therapeutic modules.

The therapeutic module has two main aspects. First, the therapeutic module stores and maintains specific biologicals and chemicals in its chambers, which are carefully mixed in unique combinations in order to provide a customized solution to unique (genetic) pathologies. In order to execute specific unique combinations of biologicals and chemicals, the iMD is able to transform its structure in order to optimize the combination of the remedies for each personalized problem. Second, the therapeutic module applies the remedy and the iMD receives feedback on the effectiveness of the solution. Since the iMD system includes integrated components and is dynamic, the initial therapy is evaluated by the diagnostic and analytical modules and the remedy is updated to improve effectiveness at solving medical problems. In particular, as the pathologies evolve over time, the iMD is able to co-evolve to solve the problems. Further, since the iMDs have typically two or more therapeutic modules installed, each iMD is able to solve two or more pathologies simultaneously. Finally, since iMDs work together as a coordinated and integrated system, the iMD network is able to solve multiple problems at the same time. These multifunctional and adaptive capabilities allow the iMD to customize complex solutions to difficult medical problems.

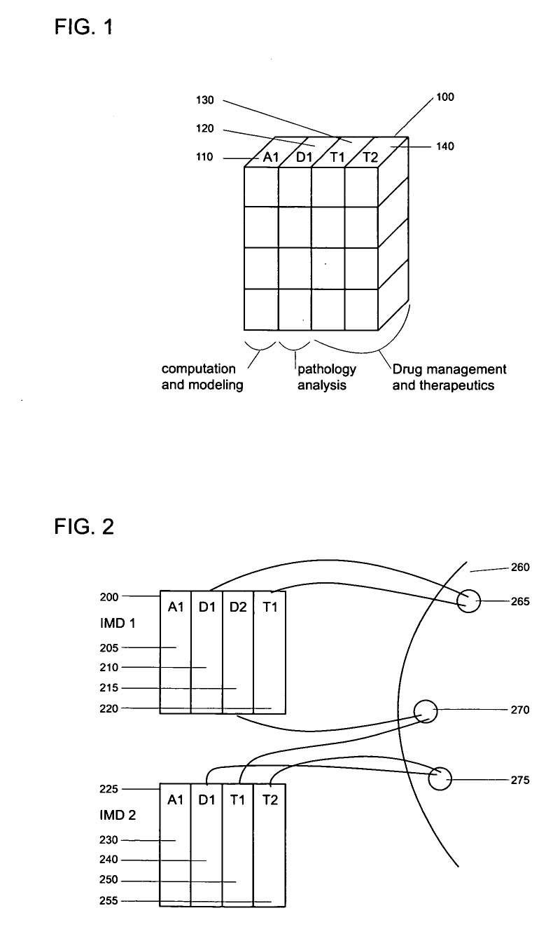

FIG. 1 shows a 3D iMD (100) with separate analytical (110), diagnostic (120) and therapeutic (130 and 140) module components. The analytical module is used for computation and modeling. The diagnostic module is used for pathology analysis. The therapeutic module is used for drug management and therapeutics.

FIG. 2 shows a network of two iMDs interacting with external pathologies simultaneously. IMD 1 (200) receives biological samples from two cell clusters (265 and 270) at D1 (210) and D2 (215). IMD 2 receives biological samples from an additional cell cluster (275) at D1 (240). The IMDs analyze the samples in the diagnostic modules, analyze the data in the analytical modules (205 and 230) and supply solution options to the therapeutic modules (220, 250 and 255). The therapeutic modules then combine specific remedies to address the specific pathologies and apply the remedies, with iMD 1 T1 supplying a remedy at 265, iMD T1 supplying a remedy at 270 and iMD T2 supplying a remedy at 275. The two iMDs work together to balance the diagnoses, analyses and remedies to solve multiple pathologies.

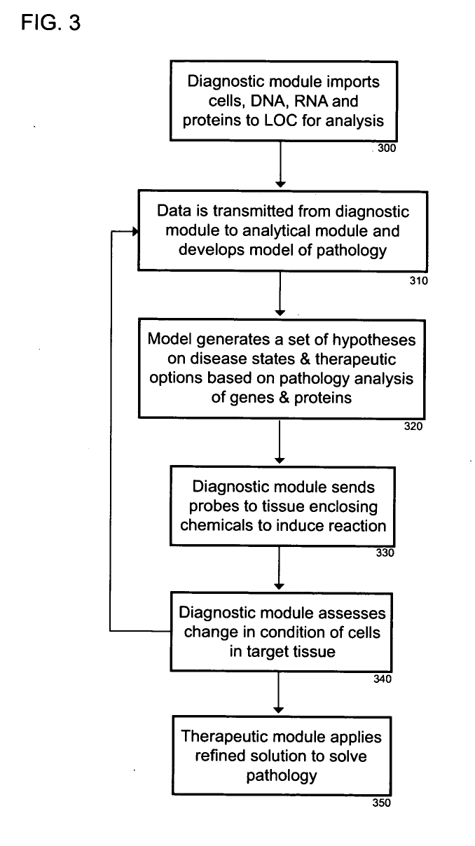

FIG. 3 shows the process of a diagnostic module collecting and analyzing biological and chemical samples. After the diagnostic module imports cells, DNA, RNA and proteins to the LOC for analysis (300), the data is transmitted from the diagnostic module to the analytical module, which develops a model of the pathology (310). The model generates a set of hypotheses on disease states and therapeutic options based on pathology analysis of genes and proteins (320). The diagnostic module sends probes to tissue enclosing chemicals to induce reaction (330) and then assesses the change in condition of the cells in the target tissue (340). The therapeutic module applies a refined solution to solve the pathology (350). In one embodiment of the invention, the diagnostic module functions are integrated into to therapeutic module.

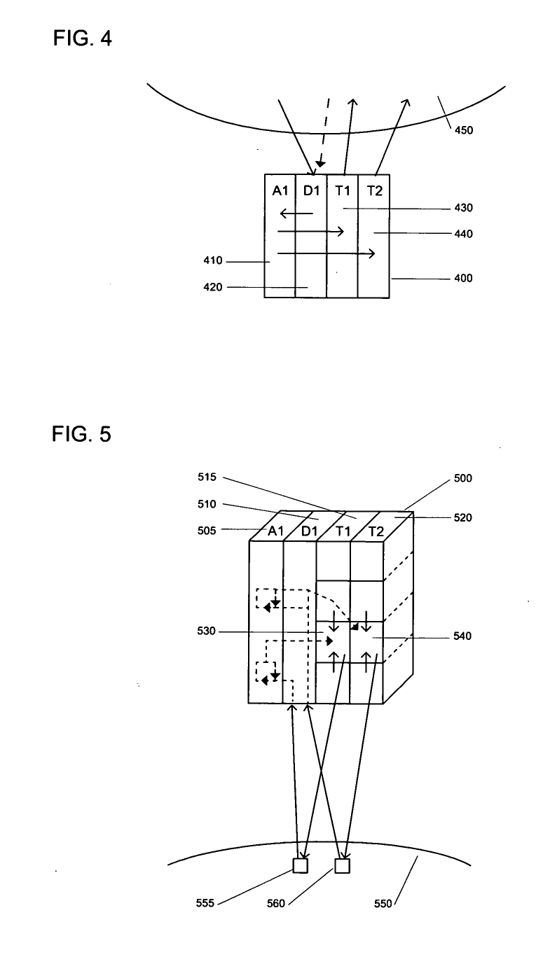

In FIG. 4, an iMD is shown interacting with a patient's tissue. After the diagnostic module (420) receives cell samples, it analyzes the samples and supplies data to the analytical module (410), which transmits the model and solution options to the therapeutic modules (430 and 440). T1 provides a therapeutic solution to the cell site location, which the diagnostic module evaluates by receiving and analyzing cell samples. The diagnostic module supplies updated data to the analytical module, which updates its model and solution options and supplies the updated remedies to T2. T2 then combines biologicals and chemicals to solve the pathology.

FIG. 5 shows a 3D iMD with internal components interacting with specific pathologies. Once cell samples (555) are received and analyzed in D1 (510) and the data is transferred to the analytical module (505) for modeling, the model sends solution options to T1. T1 develops specific remedies based on the model and applies the solution at 555. Cell samples at nearby tissue (560) are then collected by the diagnostic module, analyzed and the data sent to the analytical module. The analytical module models the new data and supplies solution option remedies to T2. T2 then supplies the remedy to the cell site at 560.

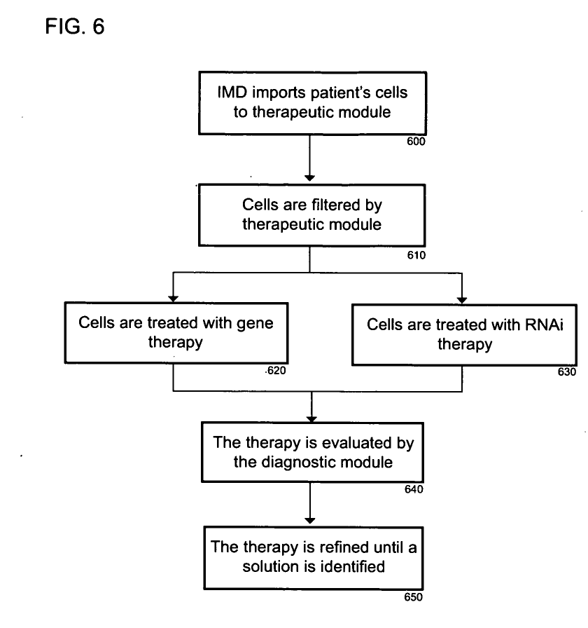

FIG. 6 shows the process of an iMD initiating therapeutic protocols. Once the iMD imports a patient's cells to the therapeutic module (600), the cells are filtered (610) and treated with gene therapy (620) or RNAi therapy (630). The therapy is evaluated by the diagnostic module (640) and refined until a solution is identified (650).

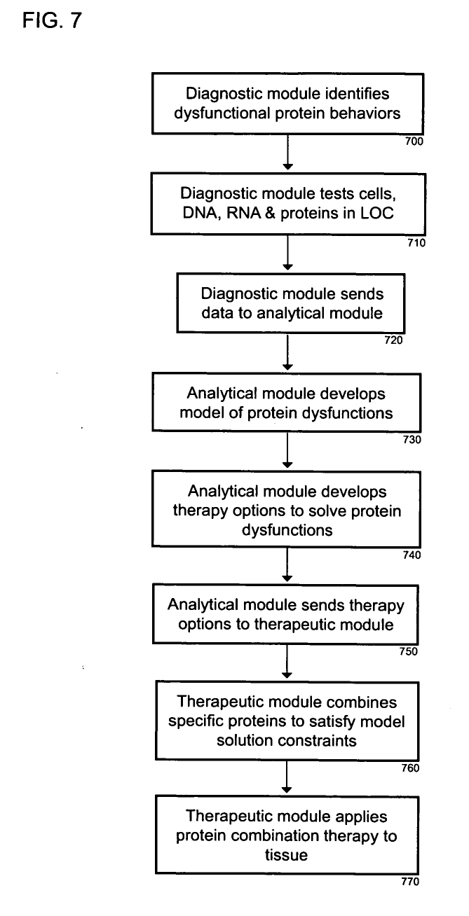

FIG. 7 shows the analytical process of assessing and applying a therapy using an iMD. The diagnostic module first identifies dysfunctional protein behaviors (700) and then tests cells, DNA, RNA and proteins in the LOC (710). The diagnostic module sends data to the analytical module (720), which develops a model of protein dysfunctions (730) as well as therapy options to solve the protein dysfunctions (740). The analytical module then sends the therapy options to the therapeutic module (750), which combines specific proteins to satisfy model solution constraints (760) and applies the protein combination therapy to tissue (770).

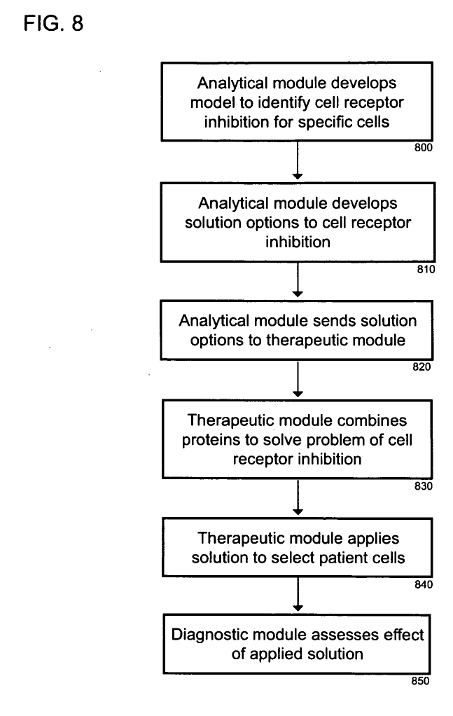

FIG. 8 shows the process of identifying cell receptor inhibition and designing a therapy using an iMD. After the analytical module develops a model to identify cell receptor inhibition for specific cells (800), it develops solution options to cell receptor inhibition (810). The analytical module sends solution options to a therapeutic module (820), which combines proteins to solve the problem of cell receptor inhibition (830). The therapeutic module applies the solution to select patient cells (840) and the diagnostic module assesses the effect of the applied solution (850).

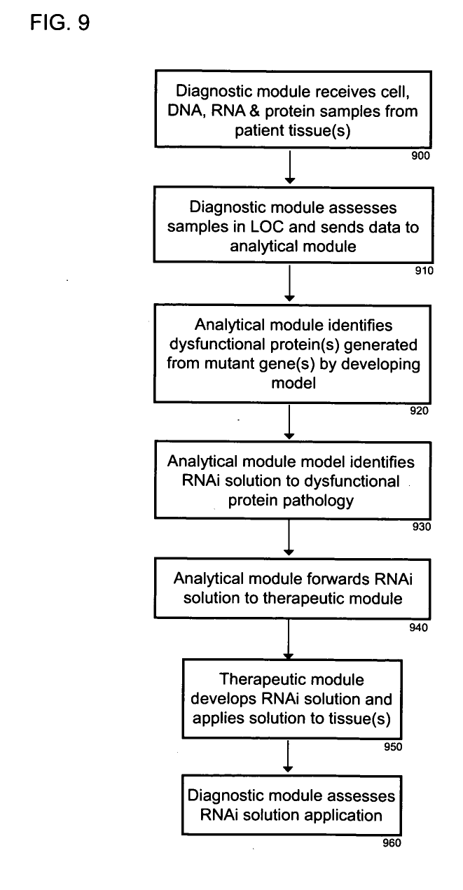

FIG. 9 shows the process of identifying dysfunctional proteins and applying RNAi solutions using an iMD. Initially, the diagnostic module receives cell, DNA, RNA and protein samples from patient tissue(s) (900) and then assesses the samples in the LOC and sends the data to the analytical module (910). The analytical module identifies dysfunctional protein(s) generated from mutant gene(s) by developing a model (920) and the model identifies RNAi solution(s) to dysfunctional protein pathology (930). The analytical module forwards the RNAi solution(s) to the therapeutic module (940) and the therapeutic module develops the RNAi solution(s) and applies the solution(s) to tissue(s) (950). The diagnostic module assesses the RNAi solution(s) application (960) and the process repeats.

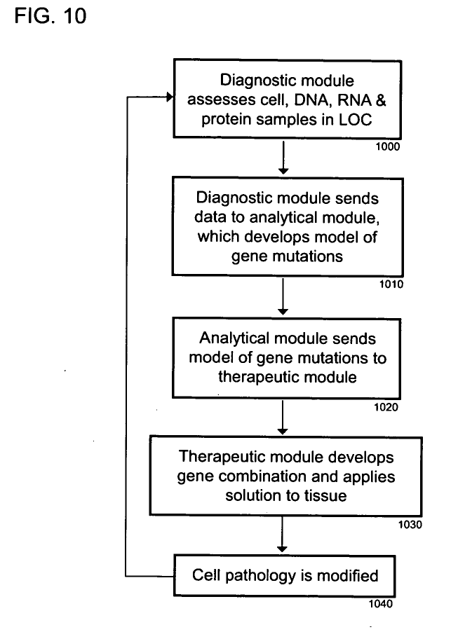

FIG. 10 shows the process of generating models of gene mutations using an iMD. The diagnostic module first assesses cell, DNA, RNA and protein samples in the LOC (1000) and then sends the data to the analytical module, which develops a model of gene mutations (1010). The analytical module sends model of gene mutations to the therapeutic module (1020) and the therapeutic module develops a gene combination and applies the solution to tissue (1030) after which the cell pathology is modified (1040) and the system repeats.

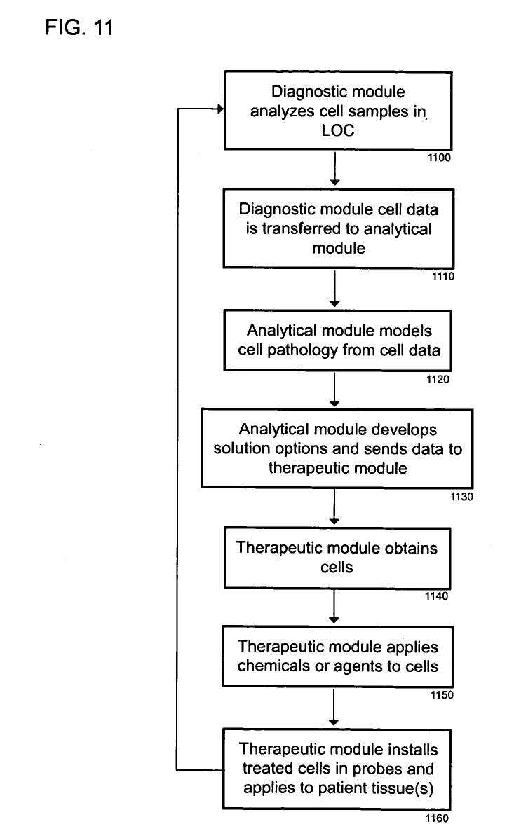

FIG. 11 shows the process of analysis and solution option generation using an iMD. The diagnostic module analyzes cell samples in the LOC (1100) and cell data is transferred to the analytical module (1110). The analytical module models cell pathology from the cell data (1120), develops solution options and sends the data to the therapeutic module (1130). The therapeutic module obtains cells (1140) and applies chemicals or agents to the cells (1150). The treated cells are installed in probes by the therapeutic module, which applies them to the patient's tissue(s) (1160) and the process repeats.

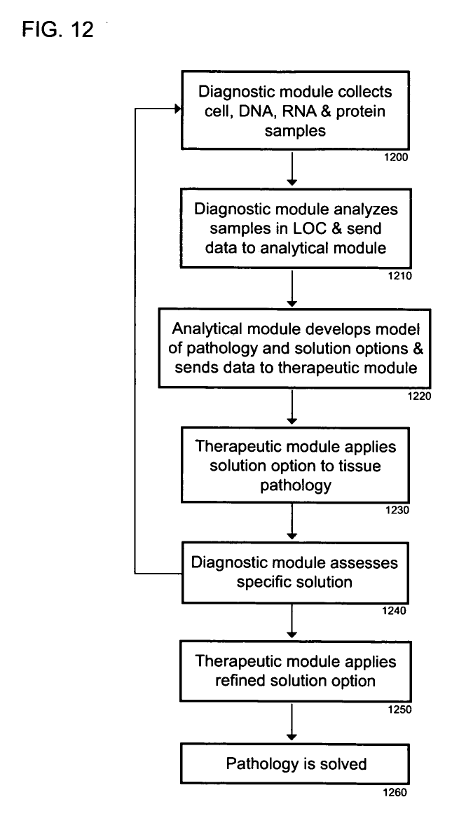

FIG. 12 shows the process of developing solution options from diagnostic data and analysis in an iMD. The diagnostic module first collects cell, DNA, RNA and protein samples (1200), analyzes samples in the LOC and sends data to the analytical module (1210). The analytical module develops a model of pathology and solution options and sends data to the therapeutic module (1220), which applies the solution option to the tissue pathology (1230). The diagnostic module assesses the specific solution (1240), which repeats the process, and the therapeutic module applies the refined solution option (1250) until the pathology is solved (1260).

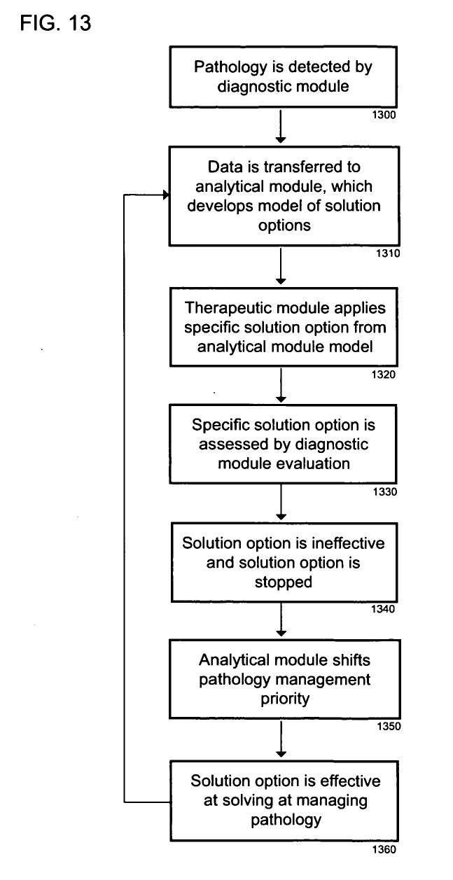

FIG. 13 shows the process of evolving a solution using the analytical and diagnostic modules of an iMD. After the pathology is detected by the diagnostic module (1300), the data is transferred to the analytical module, which develops a model of solution options (1310). The therapeutic module applies a specific solution option from the analytical model (1320) and the solution option is assessed by diagnostic module evaluation (1330). The solution option is evaluated to be ineffective and the solution option is stopped (1340). The analytical module shifts pathology management priority (1350) based on the results of the test of the application of prior solution options until a candidate solution option is effective at solving or managing the pathology (1360). The process repeats. In one embodiment of the invention, the analytical functions are integrated into the diagnostic module. In another embodiment, the analytical functions are integrated into the therapeutic module.

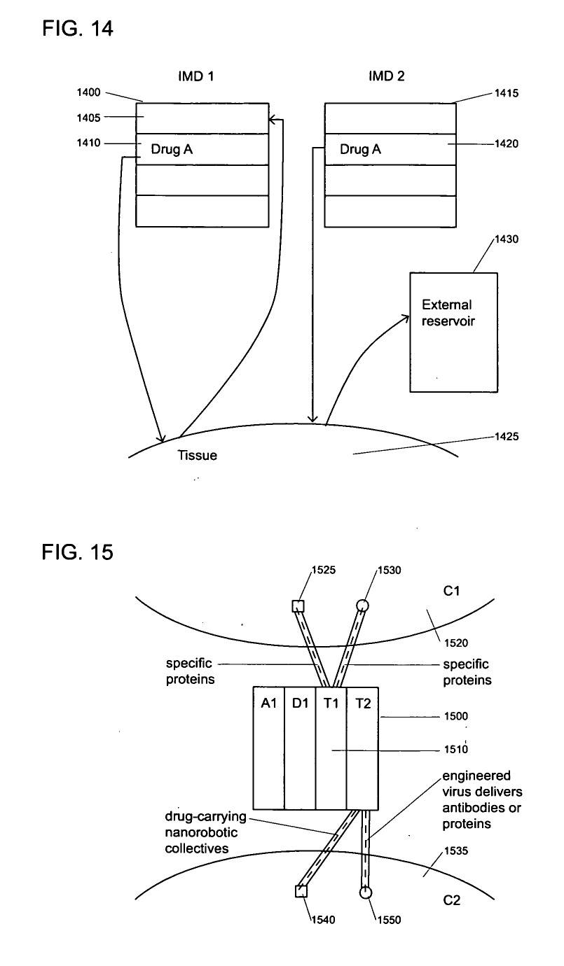

FIG. 14 shows two drugs administered by two iMDs to a tissue. IMD 1 (1400) collects and analyzes samples in the diagnostic module (1405) and analyzes the data in the analytical module before recommending the application of drug A (1410). The therapeutic module applies the drug to the tissue site (1425). In addition, the analytical module communicates with iMD 2 (1415), which administers drug B (1420) with its therapeutic module to the same tissue area. To expel diseased cells, an external reservoir (1430) is used.

FIG. 15 shows an iMD simultaneously interacting with two tissues and solving two pathologies with different therapies. The iMD (1500) therapeutic module one (1510) applies two different types of remedies to C1 (1520), including monoclonal antibodies (1525) and specific proteins (1530). At the same time, therapeutic module two applies two additional differentiated types of remedies to C2 (1535), including drug carrying nanodevices (1540) and an engineered virus delivering antibodies or proteins (1550).

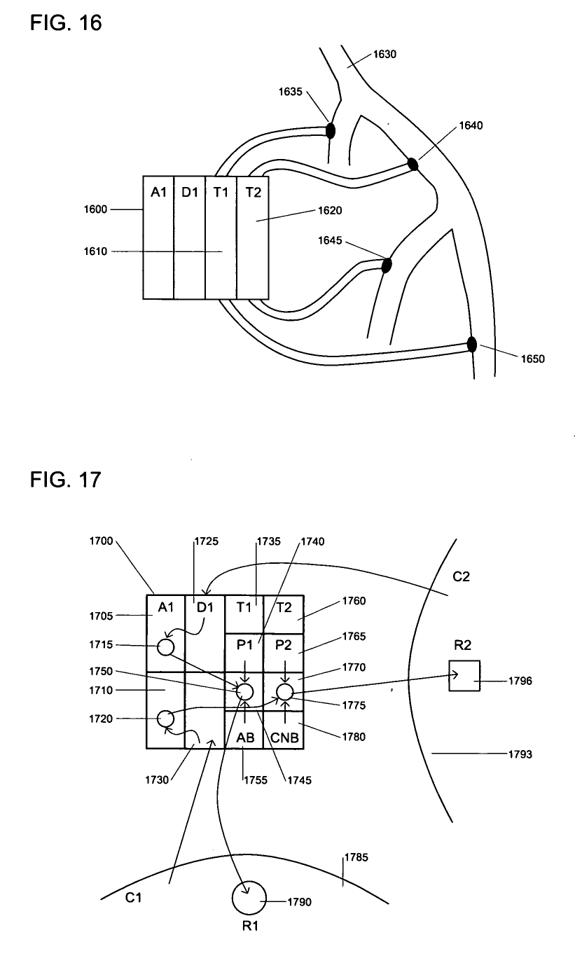

FIG. 16 shows an iMD interacting with specific tissue locations in the vasculature using vascular zip code proteins. The vascular locations that are targeted in the figure include 1635, 1640, 1645 and 1650. The iMD therapeutic modules 1 (1610) and 2 (1620) apply therapies to these locations that include proteins matched to each respective vascular zip code.

The iMD is useful for managing disease by identifying pathology solutions and applying protein therapy. In one approach, the iMD induces cells in a tissue site to manufacture proteins to solve problems. In another approach, the iMD influences healthy allied cells to manufacture proteins to apply to pathological cell lines or protein regulatory networks. The manufactured protein is extracted by the iMD and applied to solve a problem on site.

FIG. 17 shows an iMD combining proteins in different therapeutic module chambers for delivery to two different pathologies. The iMD (1700) collects cell, DNA, RNA and protein samples from C2 (1793) at the diagnostic module (1725), analyzes the samples and forwards the data to the analytical module (1715), which develops a model and solutions options. Solution options are forwarded to the therapeutic module 1 (1735), which combines proteins (1740) and antibodies (1755) in a chamber (1745) into a remedy (1750) in order to deliver to another cell site (1785) at R1 (1790). The iMD collects cell, DNA, RNA and protein samples from C1 (1785), analyzes the samples and forwards the data to the analytical module (1715), which develops a model and solutions options. Solution options are forwarded to the therapeutic module 2 (1760), which combines proteins (1765) and nanodevices (1780) in a chamber (1770) into a remedy (1775), which is applied to R2 (1796). This process is particularly useful when the pathological tissues are adjacent to healthy cells and not all cell samples are accessible to the iMD at the same time.

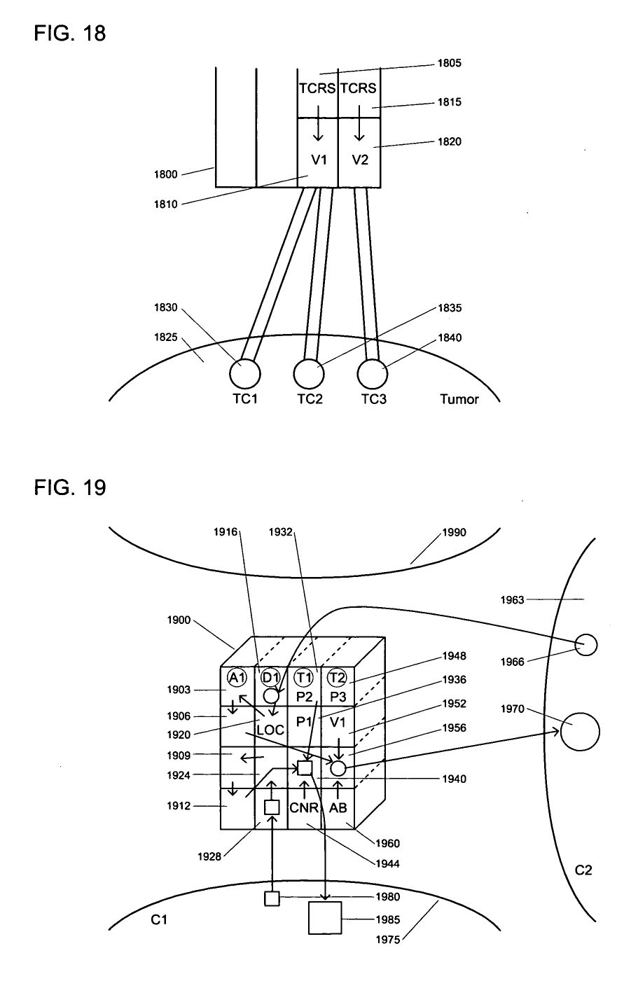

FIG. 18 shows a T cell receptor gene transferred to viruses in chambers of two therapeutic modules of an iMD to solve several pathologies. The T cell receptor genes (1805 and 1815) are inserted into the viruses (1810 and 1820) and then applied to a tumor (1825) to penetrate T cells 1, 2 and 3 (1830, 1835 and 1840) to train the cells to destroy the tumor.

FIG. 19 shows an iMD with multiple compartments in which proteins, viruses and antibodies are combined for treating two simultaneous pathologies. After cell, DNA, RNA or protein samples are collected at 1966 in tissue C2 (1963) by the diagnostic module (1916), the samples are analyzed in the LOC (1920) and the data transmitted to the analytical module (1903) for analysis and modeling (1906). The model develops solution options, which are forwarded to therapeutic module 2 (1948). T2 combines viruses (1952) and antibodies (1960) in a chamber (1956) to apply to the pathology (1970). At the same time, cell, DNA, RNA or protein samples are collected at 1980 in tissue C2 (1975) by the diagnostic module (1928), the samples are analyzed in the LOC (1924) and the data transmitted to the analytical module (1909) for analysis and modeling (1912). The model develops solution options, which are forwarded to therapeutic module 1 (1932). T1 combines proteins and nanodevices (1944) in a chamber (1940) to apply to the pathology (1985). The advantage of having two therapeutic modules is the ability to maintain two distinct therapeutic regimen at the same time to treat different pathologies.

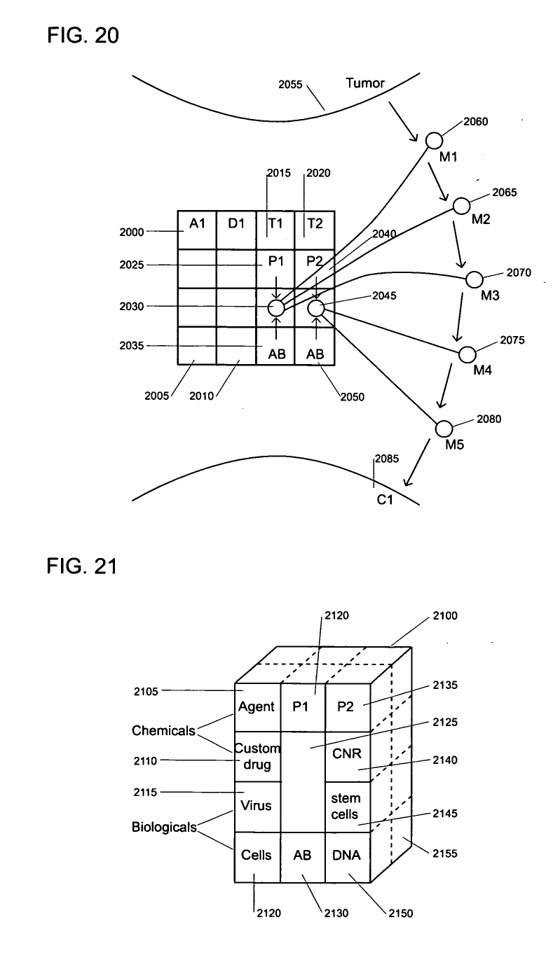

FIG. 20 shows an iMD with multiple compartments in which proteins are combined with antibodies in two therapeutic modules in order to track, attack and destroy metastatic cancer cells. Tumor cells affect other tissue when cells detach and spread or metasticize. Metasticization is a leading cause of cancer death and, if it can be controlled, will lead to management of solutions to cancer. In FIG. 20, a tumor (2055) has cells that metasticize (2060-2080) and spread to other tissue (2085). The iMD receives samples from the tumor (not shown), analyzes the samples in the diagnostic module (2010) LOC and transfers the data to the analytical module (2005) for modeling, analysis and solution option generation. The solution options are transmitted to therapeutic modules 1 (2015) and 2 (2020) and the remedies are combined from different proteins (2025 and 2040) and antibodies (2035 and 2050). The remedies are then applied to metastasized tumor cells M 1 to M5 (2060-2080) before they infect C1 (2085).

FIG. 21 shows the side view of the therapeutic module with multiple biologicals and chemicals in specific compartments. The therapeutic module combines biologicals and chemicals in central chamber(s) and then administers the combined remedy solutions to specific cells. The biological and chemical components are stored in compartments on the periphery of specific layers of the therapeutic module(s). In this embodiment of the invention, chemicals such as agents (2105) and a custom drug (2110) are stored as shown. Biologicals, which include viruses (2115), cells (2120), antibodies (2130), DNA (2150), stem cells (2145), nanodevices (2140) and different proteins (2120 and 2135) are stored as shown in separate compartments. The compartments are connected by a maze of microfluidic conduits, valves and sensors that allow the movement of the component parts to specific locations on demand. In particular, the components are moved to adjacent chambers for measurement, mixing and testing, before application to a specific disorder.

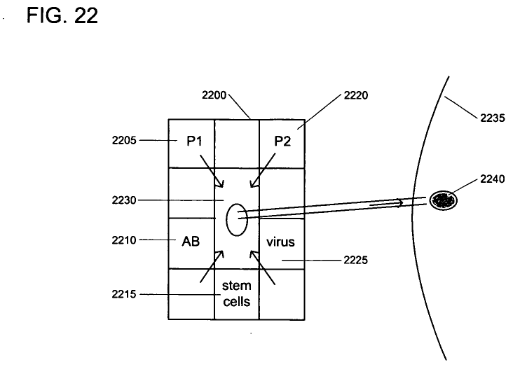

FIG. 22 shows the compartments of a therapeutic module of an iMD in which proteins, antibodies and a virus are combined and delivered to a cell site for therapy. The side view of a therapeutic module of an iMD (2200) shows proteins (2205 and 2220) in separate compartments and antibodies (2210), a virus (2225) and stem cells (2215) in separate compartments. The proteins, antibodies and viruses are combined in a specific order in the center chamber (2230) and then delivered to the specific location (2240) in the tissue (2235) for therapy.

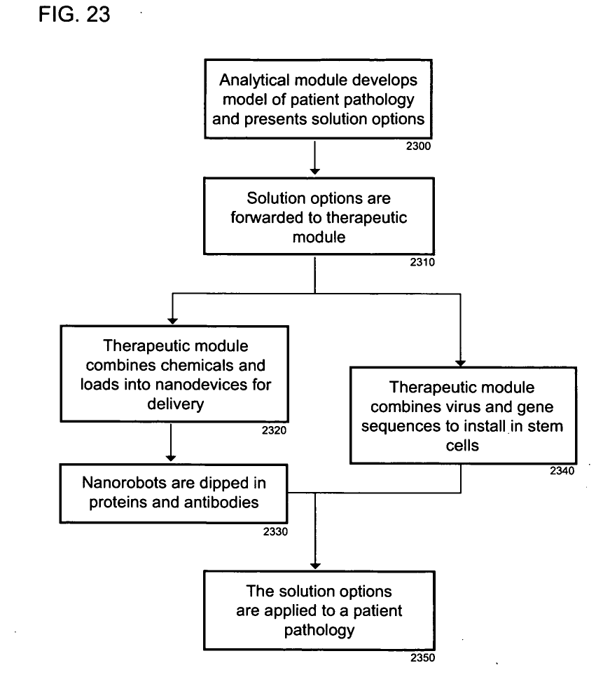

FIG. 23 shows the process of combining specific biologicals and chemicals for application to pathologies by using the therapeutic module of an iMD. After the analytical module develops a model of patient pathology and presents solution options (2300), the solution options are forwarded to the therapeutic module (2310). The therapeutic module combines chemicals and loads them into nanodevices (for delivery) (2320) and the nanodevices are dipped in proteins and antibodies (2330). The therapeutic module also combines a virus and gene sequences to install in stem cells (2340), in order to deliver the modified virus. The solution options are then applied to a patient pathology (2350).

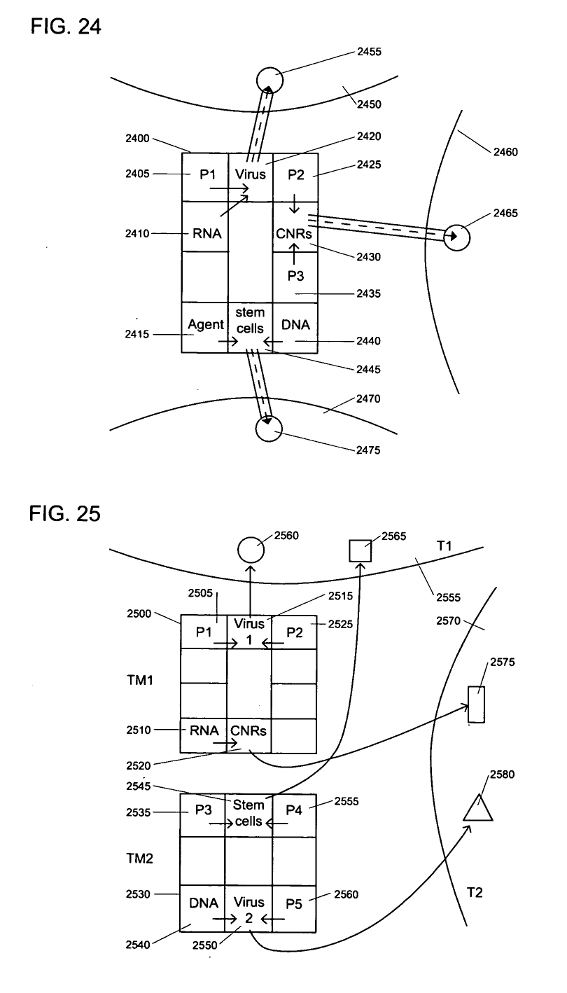

FIG. 24 shows different delivery vehicles, including a virus, nanodevices and stem cells, applied by a therapeutic module of an iMD simultaneously to different tissues. In this figure, which represents a side view of a therapeutic module of an iMD (2400), a protein (2405) and RNA (2410) are combined with a virus (2420) to apply to a tissue (2450) cell site (2455). At the same time, two proteins (2425 and 2435) are combined with nanodevices (2430) and applied to a tissue (2460) cell site (2465). The therapeutic module also combines an agent (2415) with DNA (2440) to install into stem cells (2445) to apply to a tissue (2470) cell site (2475). The capacity of the iMD to solve multiple problems with a single therapeutic module is dependent only on the capacity of the module to maintain multiple components and the interconnections with different tissues.

FIG. 25 shows two therapeutic modules solving several problems simultaneously. T1 (2500) combines two proteins (1 and 2) (2505 and 2525) with a virus 1 (2515) and apply to a cell site (2560) in tissue 1 (2555) while the same therapeutic module inserts RNA (2510) into nanodevices (2520) and applies the devices to a cell site (2575) in tissue 2 (2570). T2 (2530) combines two proteins (3 and 4) (2535 and 2555) with stem cells (2545) and applies the cells to cell site 2565 at tissue 1 (2555) while the same therapeutic module inserts DNA (2540) and a protein (5) (2560) into a virus 2 (2550) for application to cell site at 2580 in tissue 2.

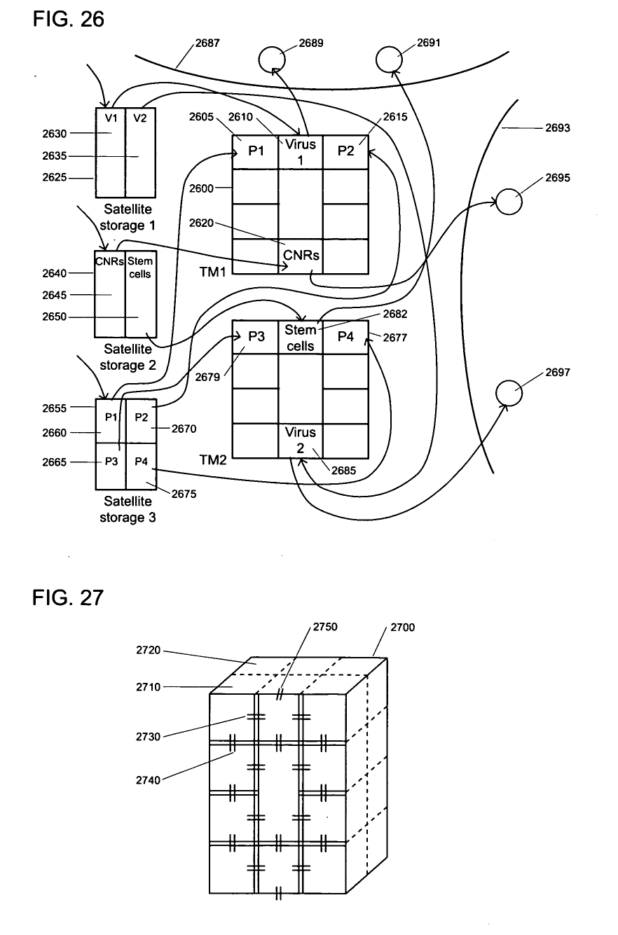

FIG. 26 shows the storage refill system mechanism in a network of iMDs. iMDs may be configured to receive and expel chemical and biological elements to and from specific compartments in order to accomplish a task. In some cases, the iMDs require refilling in order fulfill a continuous stream of therapies. For these cases, the integrated refill system is useful. The satellite storage units may be internal holding compartments or may involve external compartments that are replaced or refilled on demand. In FIG. 26, two iMD therapeutic modules (T1 and T2) (2600 and 2677) are shown with three satellite storage units (1, 2 and 3) (2625, 2640 and 2655). Each of the satellite storage units in this example is comprised of multiple compartments with at least two distinct biological or chemical components with (typically) similar composition. SS 1 is comprised of two different viruses in different compartment (V1 and V2) (2630 and 2635), SS 2 is comprised of nanodevices (2645) and stem cells (2650), while SS 3 contains four different proteins (P1, P2, P3 and P4) (2660, 2670, 2665 and 2675). Each of the satellite storage units has inputs into the storage devices to resupply the elements.

The component biological and chemical units are supplied from the satellite storage units to the therapeutic modules to refill each component as it becomes depleted. In the embodiment shown, the proteins 1 and 2 (2665 and 2615) are applied to virus 1 (2610) and transmitted to cell site 2689 in tissue at 2687. Similarly, proteins 3 and 4 (2679 and 2677) in T2 are combined with stem cells (2682) to be delivered to a cell site (2691) at tissue 2687. Proteins 1 and 2 and virus 1 are combined with nanodevices (2620) for delivery to a cell site (2695) at tissue 2693. Similarly, proteins 3 and 3 and stem cells are combined with virus 2 (2685) and applied to a cell site (2697) at tissue 2693.

FIG. 27 shows the conduit network of a therapeutic module (2700). The conduits are represented by the double lines and run through the edges of the compartments of each therapeutic module layer (2710 and 2720). The internal valves indicated at 2730 and 2740 control the flow of fluids between compartments, while the external valves indicated at 2750 and 2760 control the flow of fluids between the therapeutic module and its environment.

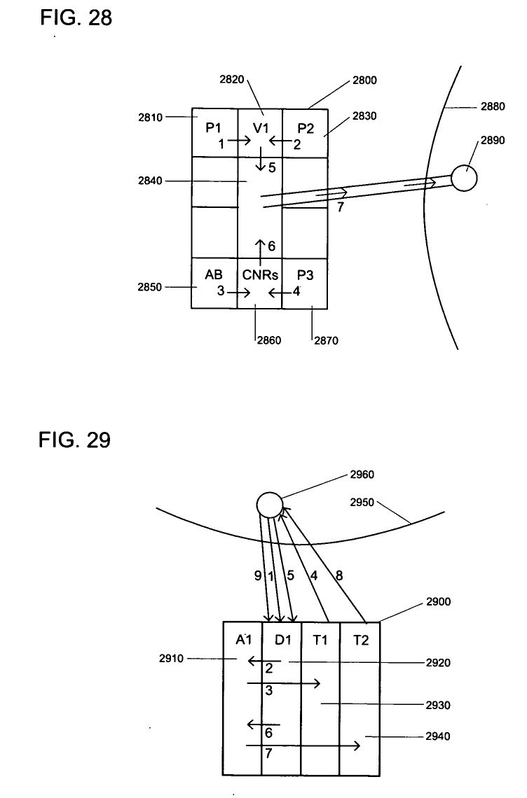

FIG. 28 shows the sequence order of combining different elements of mixing all elements for application by a therapeutic module of an iMD for solving a pathology. Protein 1 (2810) and protein 2 (2830) are combined with virus 1 (2820). Antibodies (2850) and protein 3 (2870) are then combined with nanodevices (2860). The combined nanodevices and the combined virus are then transferred to a central chamber (2840) and delivered to the cell site (2890) in tissue 2880.

FIG. 29 shows the order of a therapeutic process with feedback using multiple modules of an iMD. Once the cell samples (2960) are moved from the tissue (2950) to the diagnostic module (2920) of the iMD (2900), the LOC analyzes the samples and forwards data to the analytical module (2910), which models and analyzes the data and sends solution options to therapeutic module 1 (2930), which combines and applies a remedy to the cell site. The diagnostic module again collects cell samples from the cell site to evaluate the remedy by analyzing the cells in its LOC and forwards the data to the analytical module for analysis and modeling. The refined solution options are sent to therapeutic module 2 (2940), which combines components and delivers the therapy to the cell site for resolution of the pathology.

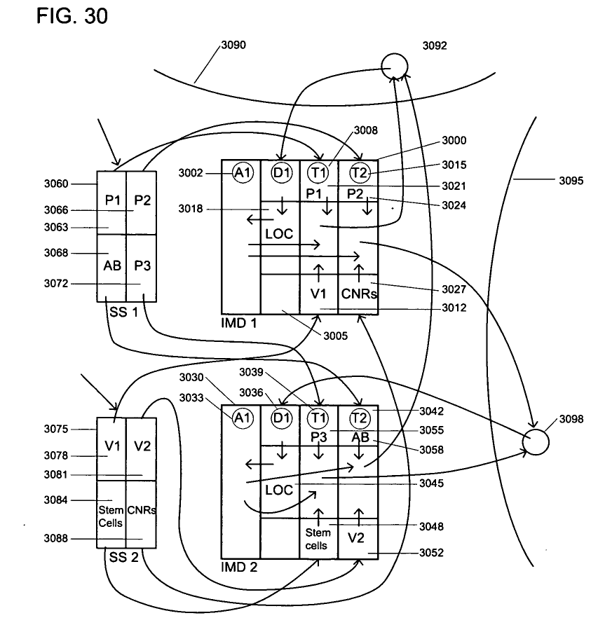

FIG. 30 shows the network operations with satellite refills and multiple parallel operations using two iMDs to solve two pathologies simultaneously. IMD 1 (3000) collects cell samples from a cell cluster (3092) site in tissue at 3090 and inputs the samples to the diagnostic module (3005) LOC (3018) which analyzes the samples and forwards the data to the analytical module (3002) for analysis, modeling and solution option generation. The solution options are transmitted to therapeutic module 1 (3008), which combines P1 and V1 (3012) and applies the combination therapy to the cell site (3092). At each stage, as the proteins and viruses of therapeutic module 1 are exhausted, or near exhaustion, the satellite storage units 1 (3060) and 2 (3075) refill the iMD components.

IMD 2 (3030) receives cell samples from a cell site (3098) in another tissue (3095) and the LOC (3045) in the diagnostic module (3036) analyzes the samples and transfers the data to the analytical module (3033) for analysis, modeling and solution option generation. The solution options are transmitted to therapeutic module 1 (3039) where protein 3 (3055) and stem cells (3048) are combined in a central chamber for application to the cell site (3098).

Information from the two iMD analytical modules is shared by allocating computational resources. When the solution options are not in iMD 1, but are in iMD 2, iMD 2 activates its therapeutic module 2, combines the antibodies (3058) and virus 2 (3052) and applies the combined solution to the cell cluster at 3092. Similarly, when the solution options are not in iMD 2, but are in iMD 1, iMD 1 activates its therapeutic module 2, combines protein 2 (3024) and nanodevices (3027) to apply the remedy to the cell cluster at 3098. This coordination of supplies among the four therapeutic modules of the two iMDs further extends the range of the system. The system is able to scale to maintain multiple iMDs continuously solving multiple pathologies in this way.

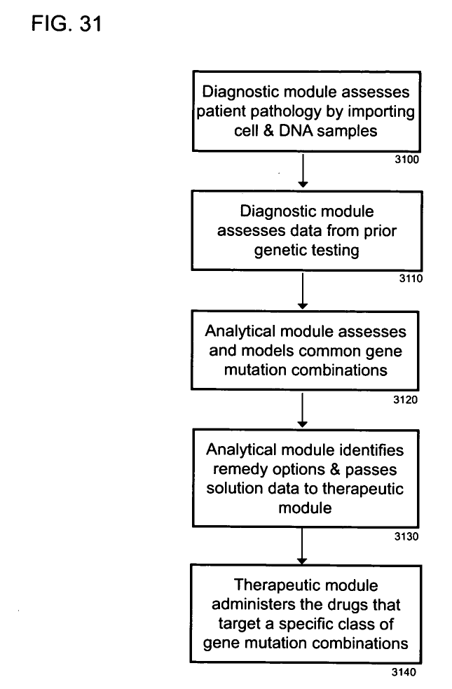

FIG. 31 shows the process of semi-personalized medicine using an iMD. After the diagnostic module assesses patient pathology by importing cell and DNA samples (3100), it assesses the data from the prior genetic testing (3110). The analytic module assesses and models common gene mutation combinations (3120), identifies remedy options and passes solution data to a therapeutic module (3130). The therapeutic module administers the drugs that target a specific class of gene mutation combinations (3140).

Drug “modules” of common proteins are collected, or the manufacture of the proteins is influenced by the iMD, to apply to specific common mutation combinations. In order to apply a common protein to similar genetic mutation combinations that cause a particular disease, multiple versions or “biosimilars” of the proteins are employed by the iMD. Since each batch of protein combinations is slightly different, assessing the range of effects of a different batch of therapies is facilitated by recording each combination in the database and comparing each batch to other similar batches. The database groups each batch into families of similar protein combinations.

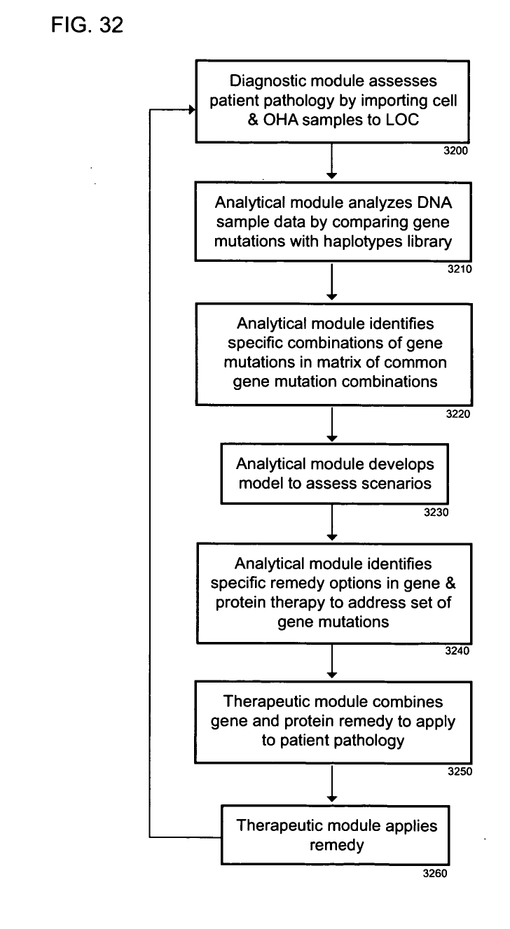

FIG. 32 shows the process of personalized medicine using an iMD. The diagnostic module first assesses a patient pathology by importing cell and DNA samples to the LOC (3200). The analytical module then analyzes the DNA sample data by comparing gene mutations with haplotypes library (3210) and identifies specific combinations of gene mutations in a matrix of common gene mutation combinations (3220). The analytical module develops a model to assess scenarios (3230) and identifies specific remedy options in gene and protein therapy to address a set of gene mutations (3240). The therapeutic module combines gene and protein remedy to apply to patient pathology (3250) and then applies the remedy (3260).

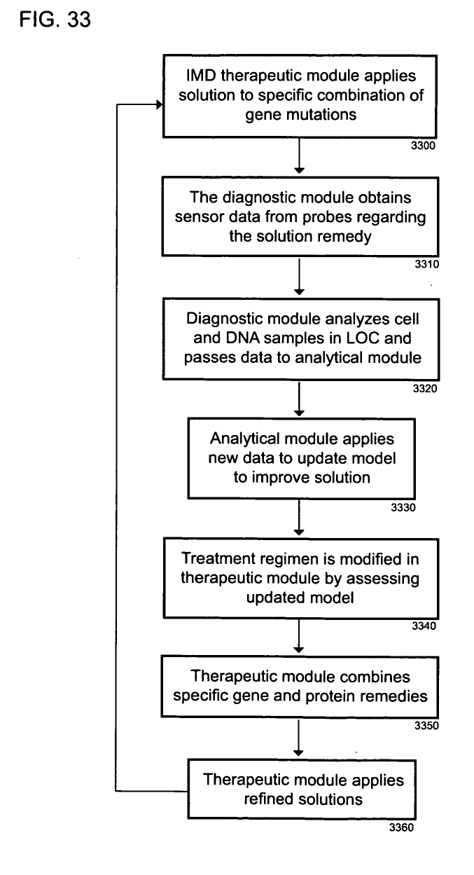

FIG. 33 shows the process of active personalized medicine using an iMD. Once the iMD therapeutic module applies a solution to a specific combination of gene mutations (3300), the diagnostic module obtains sensor data from probes regarding the solution remedy (3310), analyzes the cell and DNA samples in the LOC and passes the data to the analytical module (3320). The analytical module applies new data to update the model to improve the solution (3330). The treatment regimen is modified in the therapeutic module by accessing an updated model (3340). The therapeutic module then combines specific gene and protein remedies (3350) and applies the refined solutions (3360). The process then repeats until the pathology is managed.

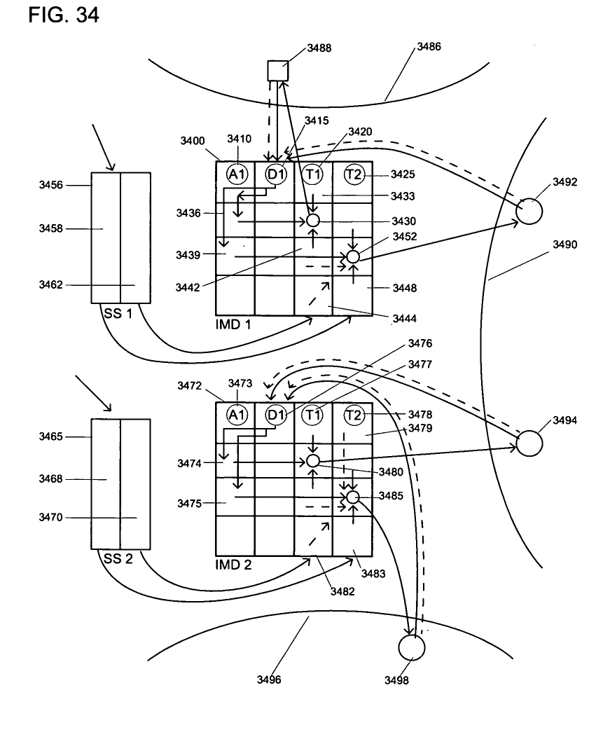

FIG. 34 shows the process of personalized medicine using two iMDs to solve several pathologies simultaneously. A cell sample from a cell cluster (3488) in tissue at 3486 is collected by the iMD 1 (3400) diagnostic module (3415). The diagnostic module analyzes the sample in its LOC and forwards the data to the analytical module (3436) for analysis, modeling and solution option generation and forwards the remedy option to therapeutic module 1 (3420), which combines specific biological and chemical elements and delivers the remedy to the cell site (3488). The same process repeats with cell samples from sites 3492 and 3494 in tissue 3490 and cell site 3498 at tissue 3496. In these cases, the iMD 1 T2 combines elements and applies the solution to 3492, iMD 2 T1 combines elements and applies the solution to 3494 and iMD 2 T2 combines elements and applies the solution to 3498. In all of these cases, as the chemical and biological elements in the therapeutic modules' compartments are exhausted, or nearly exhausted, they are refilled by the satellite storage units (3456 and 3465) as needed.

The dosage of a therapy is ascertained and refined by using feedback from the initial remedy as assessed by the diagnostic and analytical modules. Fuzzy logic algorithms are employed to refine the dosage by using statistical norms and assessing the range of patient reactions. In addition, as conditions change, and the equilibrium of the patient's pathology changes, the dosing of the remedies is modified until the therapy dose is in equilibrium.

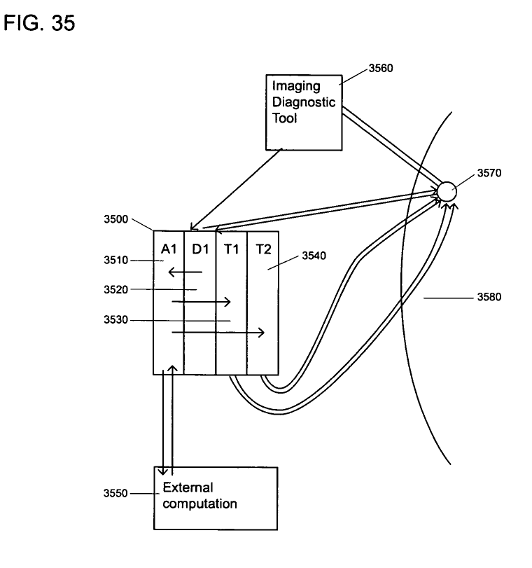

FIG. 35 shows an iMD interacting with a pathology by using external computation for analysis after using an imaging diagnostic tool. Once the pathology is identified by the imaging diagnostic tool (3560), the iMD (3500) is focused on the specific cell cluster (at 3570). The diagnostic module (3520) of the iMD collects samples, analyzes the samples in its LOC and forwards the data to the analytical module (3510), which analyzes, models and generates solution options from the data. In this example, the iMD analytical module shares computation resources with external computation (3550) to create a robust and rapid model. The solution options are transmitted to both the therapeutic modules (3530 and 3540) for construction of remedy options, which are applied to the cell cluster to solve the pathology.

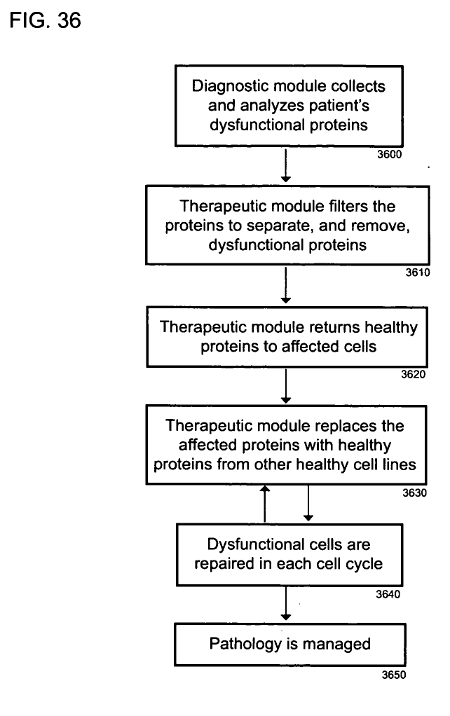

FIG. 36 shows the process of using the iMD diagnostic and therapeutic modules to acquire and filter a patient's proteins for protein replacement therapy. After the diagnostic module collects and analyzes the patient's dysfunctional proteins (3600), the therapeutic module filters the proteins to separate, and remove, dysfunctional proteins (3610). The therapeutic module returns healthy proteins to affected cells (3620) and replaces the affected proteins with healthy proteins from other healthy cell lines (3630). The dysfunctional cells are repaired in each cell cycle (3640) and the pathology is managed (3650).

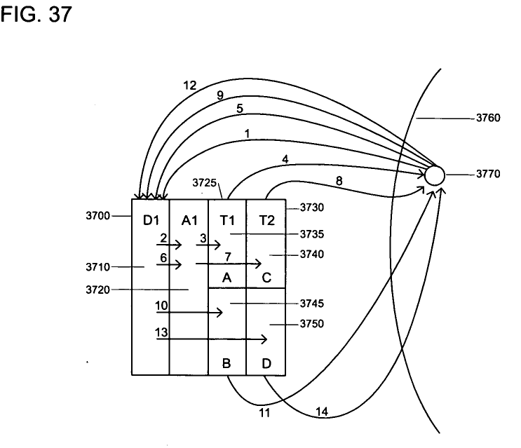

FIG. 37 shows the sequence of therapy from course-grained to fine-grained response to diagnosis and analysis by using feedback of therapy to update the analytical model to refine solutions to pathologies using the iMD. In the first state, the cell samples are collected from the cell site (3770) by the diagnostic module (3710) of the iMD (3700), analyzed in the LOC and the data are transmitted to the analytical module (3720). The analytical module models the data and develops solution options, which are transmitted to the therapeutic module 1 (3725). T1 then combines elements and forwards the therapy to the cell site (3770). This process repeats several times until the solution is refined. The feedback from each subsequent phase of the process is used to improve the model and the solution options. The pathology is ultimately managed or defeated. In another embodiment, the later phases include extensive analytical modeling for precise solution refinement.

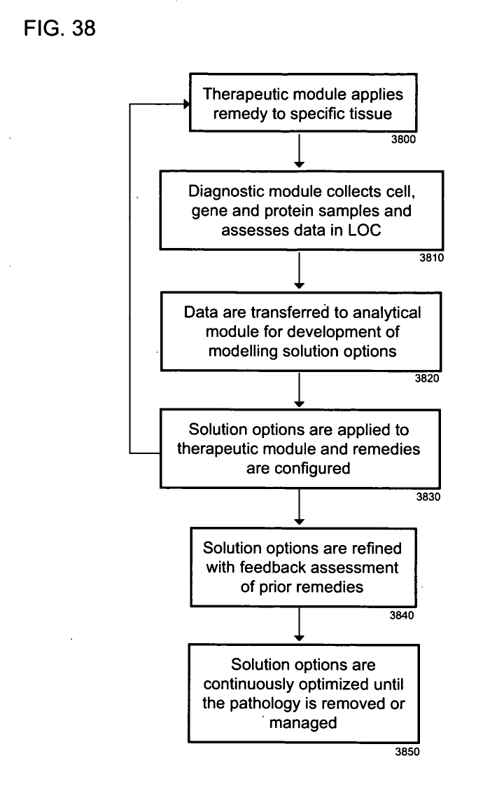

FIG. 38 shows the process of therapeutic assessment and refinement by updating model and solution options using an iMD. Initially, the therapeutic module applies a remedy to a specific tissue (3800). The diagnostic module then collects cell, gene and protein samples and assesses data in the LOC (3810). The data are transferred to the analytical module for development of modeling solution options (3820) and solution options are applied to the therapeutic module and remedies are configured (3830). This process repeats until the solution options are refined with feedback assessment of prior remedies (3840) and solution options are continuously optimized until the pathology is removed or managed (3850).

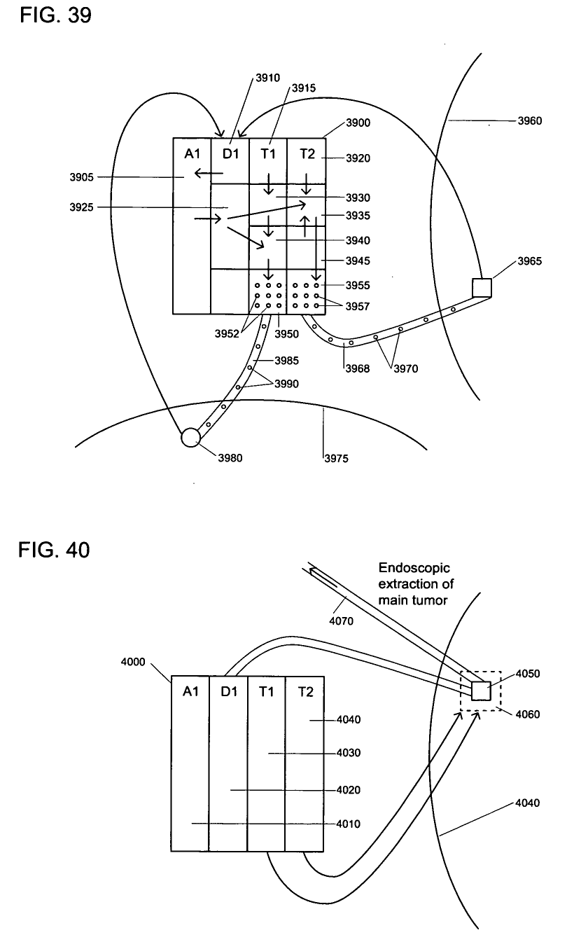

FIG. 39 shows how probes perform diagnostics and apply therapy at two cell sites simultaneously using an iMD. Probes collect cell samples from cell sites at 3965 and 3980 (in tissues 3960 and 3975) and deliver the samples to the diagnostic module (3910) for analysis in the LOC. The LOC transfers analysis data to the analytical model (3905), which analyzes, models and generates solution options, which are transmitted to the therapeutic modules (3915 and 3920). Different elements are combined to develop remedies in the therapeutic modules. The remedies (3951 and 3952) are then moved to the compartments shown at the bottom of the therapeutic modules (3950 and 3955). These remedies are then loaded onto the probes for transport (3970 and 3990) to the cell sites (3965 and 3980) by using the tubular (3968 and 3985) interconnects.

FIG. 40 shows probes from an iMD identifying a tumor, which is then extracted endoscopically, and targeting of the surrounding tissue with the therapeutic module of the iMD. The iMD may work with other surgical procedures to assist in post-operative solutions. In this example, a main tumor (4050) is extracted (4070) by a surgeon from tissue (4040). The area surrounding the tissue, marked here by 4060, is targeted by remedies of therapeutic modules 1 and 2 (4030 and 4040).

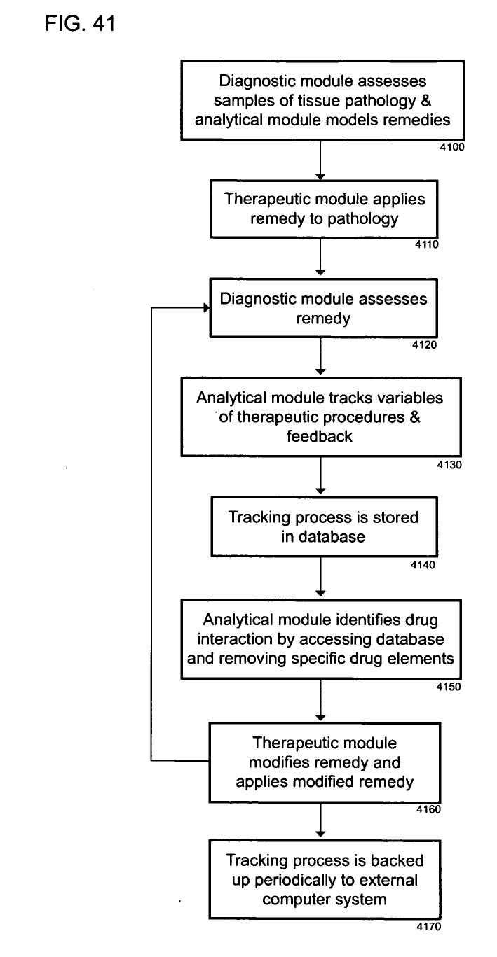

FIG. 41 shows the process of a diagnostic module and an analytical module tracking a remedy, assessing and comparing drug interactions to a database and modifying the remedies. After the diagnostic module assesses samples of tissue pathology and the analytical module models remedies (4100), the therapeutic module applies remedy to pathology (4110) and the diagnostic module assesses the remedy (4120). The analytical module tracks variables of therapeutic procedures and feedback (4130) from the environment of the reaction to the remedies and the tracking process is stored in the database (4140). The analytical module identifies drug interaction by accessing a database and removing specific drug elements (4150). The therapeutic module modifies the remedy and applies the modified remedy (4160) and the process repeats of diagnosing the remedy. The tracking process is backed up at regular intervals to an external computer system (4170).

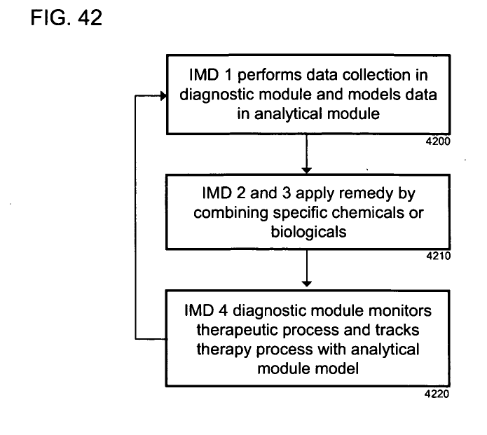

FIG. 42 shows the process of several iMDs working together to apply and track remedies to several pathologies. IMD 1 initially performs data collection in the diagnostic module and models data in the analytical module (4200). IMD 2 and iMD 3 apply the remedy by combining specific chemicals or biologicals (4210). IMD 4 diagnostic module monitors therapeutic process and tracks the therapy process with analytical module model (4220).

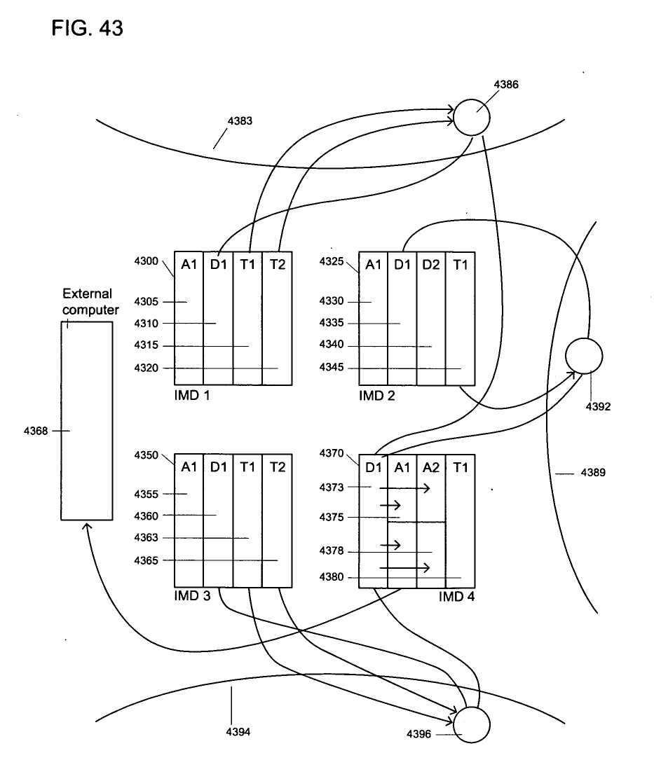

FIG. 43 shows an iMD tracking the performance of several other iMDs as they solve several simultaneous pathologies. IMD 1 (4300) solves the pathology at 4386, iMD 2 (4330) solves the pathology at 4392 and iMD 3 (4350) solves the pathology at 4396. IMD 4 (4370) tracks the performance of all three iMDs as they perform their analytical, diagnostic and therapeutic functions, with the assistance of the external computer (4368). The tracking of the three iMD performance by the fourth iMD facilitates the optimization of the coordination of the iMDs in a network and supplies excess capacity.

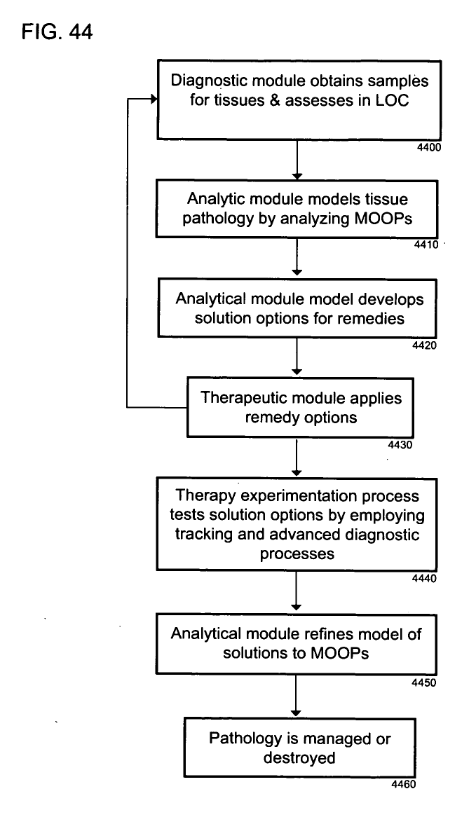

FIG. 44 shows the therapy testing and optimization process. After the diagnostic module obtains samples for tissues and assesses them in the LOC (4400) the data is forwarded to the analytical module, which models the tissue pathology by analyzing multi-objective optimization problems (MOOPs) (4410). The analytical module model develops solution options for remedies (4420) and the therapeutic module applies remedy options (4430). This process repeats. The therapy experimentation process tests solution options by employing tracking and advanced diagnostic processes (4440) and the analytical module refines the model of solutions to MOOPs (4450). The pathology is managed or destroyed (4460).

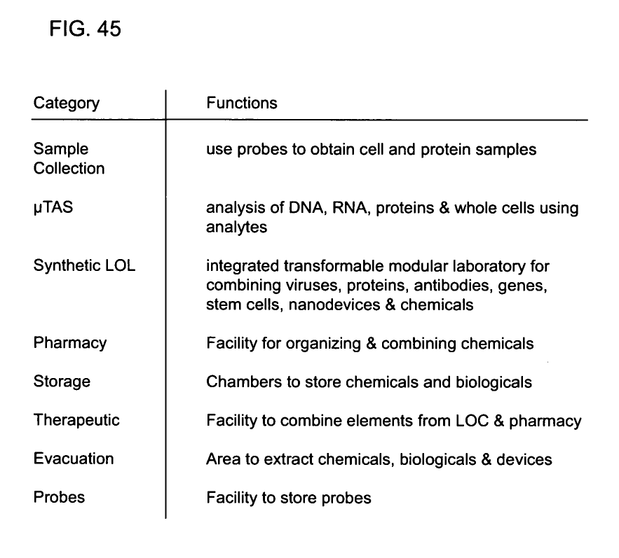

FIG. 45 shows the iMD module categories and functions.

I claim:

1. A system for operation of a medical device for therapeutics, comprising:
a set of layers of medical device components;
a set of compartments for storage of chemicals and biologicals;
a set of electrical interconnects;
a set of microfluidic components, including tubes, valves and gates;
at least one integrated circuit;
wherein the layers of components are connected by the electrical interconnects;
wherein the process is controlled by the integrated circuit;
wherein the medical device components include a set of compartments for combining chemicals and biologicals on at least one of a set of layers;
wherein the medical device components are activated after obtaining data from a medical device model for therapeutic recommendations;
wherein the medical device components are coordinated to release specific chemicals and biologicals from compartments on at least one of a set of layers through the microfluidic components in specific measured doses according to the model recommendations;
wherein the chemicals and biologicals are combined in a chamber of the medical device module in one of a set of layers; and
wherein the resulting therapeutic combination is transmitted to a cell site in a patient.

2. A system for operation of a medical device for therapeutics, comprising:
a therapeutic module consisting of at least two layers;
a diagnostic module consisting of at least two layers, including a lab-on-a-chip (LOC);
an analytical module consisting of a system-on-a-chip (SoC);
a set of compartments for storage of chemicals and biologicals;
a set of electrical interconnects;
a set of microfluidic components, including tubes, valves and gates;
at least one integrated circuit;
wherein the layers of components are connected by the electrical interconnects;
wherein the process is controlled by the integrated circuit;
wherein the diagnostic module tests cell, DNA, RNA and protein samples in the LOC and transfers the test data to an analytical module;
wherein the analytical module uses the SoC to model the biological data and transfers the data to the therapeutic module;
wherein the medical device components include a set of compartments for combining chemicals and biologicals on at least one of a set of layers;
wherein the medical device components are activated after obtaining data from a medical device model for therapeutic recommendations;
wherein the medical device components are coordinated to release specific chemicals and biologicals from compartments on at least one of a set of layers through the microfluidic components in specific measured doses according to the model recommendations;
wherein the chemicals and biologicals are combined in a chamber of the medical device module in one of a set of layers;
wherein the resulting therapeutic combination is transmitted to a cell site in a patient;
wherein the diagnostic module obtains cell, DNA, RNA and protein samples from the cell site after the initial administration of the drugs;
wherein the diagnostic module tests the samples in the LOC and sends the resulting data to the analytical module;
wherein the analytical module updates the model and sends solution options to the pathology to the therapeutic module;
wherein the therapeutic module combines a new set of chemicals and biologicals according to the revised model in a chamber on one of its layers; and
wherein the resulting revised therapeutic combination is transmitted to a cell site in a patient.
